(19) United States
(12) Patent Application Publication
(10) Pub. No.: US 2026/0139262 A1
(43) Pub. Date: May 21, 2026

(54) PROTEIN NANOPARTICLE AND METHODS OF USING SAME

(71) Applicant: THE STATE OF ISRAEL, MINISTRY OF AGRICULTURE & RURAL DEVELOPMENT, AGRICULTURAL R, Rishon Lezion (IL)

(72) Inventors: Guy MECHREZ, Kiryat Ono (IL); Avital BENHAIM, Rosh Haain (IL); Ziv SHPIGELMAN, Rishon LeTzion (IL); Reut FELDBAUM, Raanana (IL)

(21) Appl. No.: 19/322,884

(22) Filed: Sep. 9, 2025

Related U.S. Application Data
(63) Continuation of application No. PCT/IL2024/050257, filed on Mar. 10, 2024.
(60) Provisional application No. 63/451,046, filed on Mar. 9, 2023.

(51) Int. Cl. C12N 15/82

(52) U.S. Cl. CPC C12N 15/8206

(57) ABSTRACT

The present invention is directed to hybrid casein nanoparticles comprising a polynucleotide bound thereto, methods of manufacturing thereof and use thereof such as for intracellular delivery of polynucleotides to a plant.

### CROSS REFERENCE TO RELATED APPLICATIONS

This application claims the benefit of priority to U.S. Provisional Patent Application No. 63/451,046 filed Mar. 9, 2023, entitled “PROTEIN NANOPARTICLE AND METHODS OF USING SAME” the contents of which are incorporated herein by reference in their entirety.

### FIELD OF THE INVENTION

The present invention is in the field of agricultural compositions, and specifically to formulations for delivery of agriculturally active agents, such polynucleotides for various applications, including, but not limited to gene editing, gene silencing, or others.

### BACKGROUND

Successful delivery of DNA and genetic engineering of the plant genome will enhance the plant's ability to produce invaluable compounds and adapt to climate change. Intracellular delivery peptides and proteins rely on the endocytic pathway as the paramount uptake mechanism. Protein-mediated DNA delivery suffers from its entrapment inside lysosome and endosome. Gene and protein delivery into intact plant cells has been reported with nanoparticles as silica, carbon nanotubes peptides, and clay. Only limited success has been achieved using cell-penetrating peptides (CPPs) based approaches.

Understanding the limitations of protein nanoparticle (PNP)-mediated delivery of DNA through the plant cell wall and membrane is important for biological and agriculture applications.

### SUMMARY

Here, the inventors characterize the transformation efficiency of DNA deposited on PNPs with different charges. The inventors found that the formation of some extent condensed DNA on the PNPs surface is a necessary condition for DNA expression. The present study offers a strategy for efficient DNA delivery to plants without vacuolar degradation.

According to some embodiments, there is provided a composition comprising a plurality of particles, wherein: each of the particle plurality of particles comprises a casein nanoparticle and a polynucleotide bound to the casein nanoparticle; the plurality of particles are characterized by: an average zeta potential below 0 mV when measured at a pH of about 5; and by an average particle size ranging between 20 nm and about 300 nm.

In some embodiments, the plurality of particles is characterized by an average zeta potential of between −1 and −50 mV.

In some embodiments, the plurality of particles is characterized by an average particle size ranging between about 100 nm and about 200 nm, as determined by DLS.

In some embodiments, a weight per weight ratio between the polynucleotide and the casein nanoparticle within the composition is between 0.1:1 and 2:1.

In some embodiments, bound is via electrostatic interactions.

In some embodiments, the polynucleotide comprises RNA, DNA, or both.

In some embodiments, the DNA comprises an expression vector or a plasmid.

In some embodiments, the plurality of particles is characterized by an average zeta potential of between −5 and −30 mV.

In some embodiments, the composition of the invention further comprising an agriculturally acceptable carrier.

In some embodiments, the composition of the invention being formulated for administration by spraying, drenching, dipping, soaking, injecting, or any combination thereof.

In some embodiments, a concentration of the plurality of particles within the composition is between 0.1 and 10 mg/ml.

In another aspect, the composition comprises a plurality of particles, wherein each of the plurality of particles comprises a casein nanoparticle and a polynucleotide bound to the casein nanoparticle; the plurality of particles are characterized by: an average zeta potential below 0 mV when measured at a pH of about 4.5; and by an average particle size ranging between 20 nm and about 300 nm.

In some embodiments, plurality of particles is characterized by an average zeta potential of between −1 and −50 mV.

In some embodiments, plurality of particles is characterized by an average particle size ranging between about 200 nm and about 250 nm, as determined by DLS; and wherein the casein nanoparticle is a non-crosslinked particle.

In some embodiments, a weight per weight ratio between the polynucleotide and the casein nanoparticle within the composition is between 0.1:1 and 2:1, and wherein the plurality of particles consists essentially of casein and the polynucleotide, including any salt thereof.

In some embodiments, the polynucleotide comprises RNA, DNA, or both; and wherein the polynucleotide is bound to an outer surface of the casein nanoparticle.

In some embodiments, the plurality of particles are characterized by an average zeta potential of between −5 and −30 mV.

In some embodiments, the composition is an agricultural composition further comprising an agriculturally acceptable carrier.

In some embodiments, the composition is formulated for administration by spraying, drenching, dipping, soaking, injecting, or any combination thereof.

In some embodiments, a concentration of the plurality of particles within the composition is between 0.1 and 10 mg/ml.

In another aspect, there is provided a method for delivering a polynucleotide into a cell of a plant, comprising contacting the plant or a part of the plant with an effective amount of the composition of the invention, thereby delivering the polynucleotide to the cell of the plant.

In some embodiments, the contacting is by a method selected from: injecting, spraying, drenching, dipping, soaking, or any combination thereof.

In some embodiments, the method is for modifying the expression of at least one gene within the plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1G include micrographs and graphs showing internalization and expression vs. time of CNPs/DsRed plasmid conjugates in the N. benthamiana cells. Internalization vs. time of the CNPs\6-AF\DsRed plasmid (pH 4.5, +13.0 mV, 214 nm) conjugates along with the expression vs. time of the DsRed plasmid were investigated by infiltration of the CNPs into N. benthamiana leaves. 1A Schematic illustration of the confocal microscopy characterization of the treated leaves vs. time. 1B Internalization of the CNPs/6-AF/DsRed plasmid conjugates and the expression of the DsRed plasmid at different time intervals. 1C1a-1C3e Fluorescent confocal microscopy micrographs at 1 h 1C1a-e, 24 h 1C2a-e, and 48 h 1C3a-e post infiltration. a-bright field, b-red fluorescence (DsRed), c-green fluorescence (CNPs\6-AF), d-blue fluorescence (chloroplast autofluorescence), e-merge. 1D 3D visualization confocal microscopy micrographs, 48 hrs. after infiltration shows the delivery of DsRed plasmid into a plant cell by the CNPs followed by its expression. 1E Z-stack of 3D images after 48 hrs shows that both the CNPs and the DsRed protein are located inside the nucleus, the yellow represent the overlay of the green and the red channels. IF Green fluorescence intensity analysis of confocal images: ANOVA F-test yielded p=0.0005. Pairwise comparison of 1 h, 24 h and 72 h by contrast t-tests. 1G Red fluorescence intensity analysis of confocal images: t-test comparing 24 h to 48 h yielded p=0.0062.

FIGS. 2A-2E include graphs and images showing hydrodynamic diameter distribution and zeta potential distribution curves obtained with DLS analysis of the following CNPs depressions: CNPs (pH 7), CNPs/6-AF (pH 7), CNPs/6-AF (pH 4.5), and CNPs/6-AF (pH 2.7). 2A Zeta potential distribution curves of the CNPs dispersions. The zeta potential of the CNPs was varied by altering the pH at three different values of 2.7, 4.5, and 7.0. 2B Hydrodynamic diameter distribution curves of the CNPs dispersions. 2C Zeta potential distribution curves of the CNPs/6-AF/DsRed plasmid (pH 4.5) conjugates at three different CNPs: DsRed plasmid ratios. 2D Hydrodynamic diameter distribution curve of the CNPs/6-AF/DsRed plasmid conjugate at pH 4.5, utilizing the highest plasmid concentration of 30 ng/μl. 2E. gel agarose electrophoresis (1%) assay for CNPs/6-AF/DsRed plasmid conjugates (2 mg/mL CNPs/6-AF) at plasmid concentrations of 10, 20, 30, 40 and 50 ng/μl.

FIGS. 3A-3D include micrographs and images showing internalization of CNPs\6-AF (pH 4.5, 13.0 mV) into N. benthamiana cells. Schematic illustrations of cell anatomy demonstrate the challenges of CNP delivery to plant cells. CNPs\6-AF (pH 4.5, +13.0 mV, 214 nm) were infiltrated into the leaves of N. benthamiana and incubated for 2 hr. The concentration of CNPs in the infiltrated dispersion was varied to study the in influence of the CNP concentrations on their cellular internalization. 3A1-A5 Cross-sectional analysis of the treated N. benthamiana leaves by confocal fluorescent microscopy. The internal part of the cells was detected by the red chloroplast autofluorescence, which was converted to blue for convenience and differs from the green fluorescence of the CNPs/6-AF. The scale bars are 20 μm. Numbers 1-5 indicate different concentrations of CNPs, 0.2, 0.4, 0.8, 2 and 6 mg/ml, respectively. 3B Snapshots of the treated leaves. 3A6 Low magnification confocal fluorescent microscopy micrographs of the injected sections of the leaves (2 mg/mL CNPs). 3C1-3C4 high magnification, confocal fluorescent microscopy micrographs of the injected sections of the leaves (2 mg/mL CNPs). 1-bright field, 2-blue fluorescence (chloroplast autofluorescence), 3-green fluorescence (CNPs\6-AF), e-merge. 3D1-3D4 confocal fluorescent microscopy micrographs of the injected sections of the leaves (2 mg/mL CNPs) with nucleus labeled by Hoechst. 1-bright field, 2-blue fluorescence (chloroplast autofluorescence), 3-green fluorescence (CNPs\6-AF), e-Hoechst.

FIGS. 4A-4G include graphs, micrographs and illustrations showing internalization of CNPs\6-AF (pH 4.5, 13.0 mV) into N. benthamiana cells. Cryo-scanning electron microscopy micrographs of N. benthamiana cytoplasm and plant organelle of 4A1-4A4 non-treated leaves (A1-A2 cytoplasm and A3-A4 plant organelle of N. benthamiana cell at different magnification) and 4B1-4B4 of treated leaves (B1-B2 cytoplasm and B3-B4 plant organelle of N. benthamiana cell at different magnification). 4C Schematic illustration showing the degradation of CNPs/6-AF (pH 4.5, +13.0 mV, 214 nm) by NtMMP1. 4D1-4D6 Confocal images capturing the transformation of undispersed CNPs/6-AF from clusters to degradable nanoparticles over time in the presence of MMP-7 (the numbers 1-6 correspond to time intervals of 2 sec., 5 min., 10 min, 20 min, 30 min and 40 min., respectively). 4E DLS analysis of disperses CNPs/6-AF in the presence of MMP-7 reveals a size decrease. 4F Absorbance spectra of CNPs, indicating a prominent wavelength peak at 290 nm. 4G Absorbance spectra of CNPs, alongside those of CNPs with 5 ng/μl and 10 ng/μl MMP-7, demonstrate a consistent absorbance for CNPs at 290 nm wavelength. However, a decline in absorbance is observed in the presence of MMP-7, indicating a dose-dependent effect.

FIGS. 5A-5G include graphs, micrographs and illustrations showing internalization of CNPs\Hoechst\DNA plasmid (pH 4.5, 13.0 mV) to N. benthamiana cells vs. time of the CNPs\Hoechst\DNA plasmid conjugates (pH 4.5, 13.0 mV) along with the degradation vs. time of the DsRed plasmid were investigated by infiltration of the conjugate into N. benthamiana leaves. 5A Schematic illustration showing the Hoechst dye structure and its binding to the major and minor grooves of the DNA helix. 5B Schematic illustration of the CNPs\Hoechst\DNA plasmid conjugates and CSLM images of the green (CNPs) channel, blue (Hoechst) channel and merged image exhibit the successful absorbance of the DNA on the CNPs 5C Schematic illustration demonstrating the internalization of CNPs\Hoechst\DNA plasmid conjugates, highlighting potential pathways of the CNPs and the plasmid within the cytoplasm and the nucleus based on CSLM images at different time intervals: 1 h (5C1a-5C1c), 24 h (5C2a-5C2c) and 48 h (5C3a-5C3c) post-conjugates infiltration, following after the localization of the conjugates over time the presence of each entity. A-6AF-green fluorescence, b-Hoechst staining, c-merged. 5D 3D visualization confocal microscopy micrographs, 1 h. after infiltration shows the delivery of CNPs\Hoechst\DNA plasmid into a plant cell and the colocalization areas as well demonstrate the large surface area of the CNPs compare to the DNA plasmid. 5E Quantitative analysis of colocalization between CNPs and Hoechst\DNA depending on time (1 h, 24 h and 48 h) using Manders' coefficient (m₁ and m₂) calculated by JACoP plugin in Fiji-ImageJ Software® ANOVA F-test yielded p=X hilari Pairwise comparison of 1 h, 24 h and 48 h by contrast t-tests. 5F Green fluorescence intensity analysis of confocal images. Pairwise comparison of 1 h, 24 h and 48 h by contrast t-tests. 5G blue fluorescence intensity analysis of confocal images: t-test comparing 24 h to 48 h.

FIGS. 6A-6C include micrographs and illustrations showing delivery of DsRed plasmid into N. benthamiana cells by CNPs\6-AF (pH 4.5, +13.0 mV, 214 nm). CNPs\6-AF\DsRed plasmid (pH 4.5, +13.0 mV, 214 nm) electrostatic conjugates were prepared in a 24 h reaction at four different CNPs: DsRed plasmid ratios of 1:0.001, 1:0.003, 1:0.005, and 1:0.01, respectively. 6A Schematic illustration of injection by picking with a pipette tip on the abaxial side of the leaf. 6B1a-6B3e Confocal fluorescent microscopy micrographs of N. benthamiana leaves 24 h post infiltration. 6B1a-e present 6AF-green fluorescence, 6B2a-e present DSRed red fluorescence, 6B3a-e present-merged; a-DsRed plasmids without CNPs, b-CNP: DsRed plasmid=1:0.001, c-CNP: DsRed plasmid=1:0.003, d-CNP: DsRed plasmid=1:0.005, e-CNP: DsRed plasmid=1:0.01. 6C 3D Z-stack micrographs of conjugates with a CNP: DsRed plasmid ratio of 1:0.01. The scale bars are 10 μm in all micrographs.

### DETAILED DESCRIPTION

According to some embodiments, there is provided a composition comprising a plurality of particles, wherein each particle of the plurality of particles comprises: a casein nanoparticle and a polynucleotide bound to the casein nanoparticle; wherein the plurality of particles are characterized by: a negative average zeta and by an average particle size ranging between 20 nm and about 300 nm.

In some embodiments, the plurality of particles of the invention are characterized by an average zeta potential ranging between −50 and −1 mV, between −50 and −7 mV, between −50 and −10 mV, between −45 and −10 mV, between −40 and −10 mV, between −45 and −5 mV, between −45 and −8 mV, between −40 and −5 mV, between −40 and −8 mV, between −25 and −5 mV, between −25 and 5 mV, between −20 and −10 mV, between −20 and 0 mV, between −30 and −7 mV, between −20 and −1 mV including any range in between, when the zeta potential is measured at a pH between about 4 and about 5, such as 4.5.

In some embodiments, the casein NP comprises casein aggregates or colloids. In some embodiments, the casein NP comprises a plurality (e.g. 2, 10, 20, 100, or any range between) of aggregated casein molecules. In some embodiments, the casein NP is substantially devoid of a void core. In some embodiments, the casein NP is a non-hollow nanoparticle. In some embodiments, the polynucleotide is bound to an outer surface of the casein NP facing the ambient. In some embodiments, the polynucleotide is electrostatically bound to the outer surface. In some embodiments, the polynucleotide is adsorbed to the outer surface of the casein NP.

In some embodiments, at least 70%, at least 80%, at least 90%, or between 70 and 100%, between 70 and 95%, between 70 and 90%, between 80 and 90%, between 80 and 95% of the plurality of particles of the invention are characterized by an average particle size ranging between about 50 and about 300 nm, between about 100 and about 250 nm, between about 80 and about 150 nm, between about 80 and about 200 nm, between about 200 and about 300 nm, between about 200 and about 250 nm, including any range in between.

In some embodiments, at least 70%, at least 80%, at least 90%, or between 70 and 100%, between 70 and 95%, between 70 and 90%, between 80 and 90%, between 80 and 95% of the casein NPs of the invention (i.e. positively charged casein NPs, as disclosed hereinbelow) are characterized by an average particle size ranging between about 50 and about 300 nm, between about 50 and about 250 nm, between about 100 and about 250 nm, between about 100 and about 230 nm, between about 80 and about 150 nm, between about 80 and about 200 nm, between about 200 and about 300 nm, between about 200 and about 250 nm, including any range in between.

In some embodiments, “average particle size” refers to an average a diameter of the particles determined by DLS.

In some embodiments, the plurality of particles of the invention consist essentially of casein nanoparticles and polynucleotide, wherein the casein nanoparticles and the polynucleotide are directly bonded. In some embodiment, the plurality of particles of the invention are non-covalently bonded. In some embodiments, the nanoparticles and the polynucleotide are electrostatically bonded. The term “electrostatically bonded” refers to an interaction between a negatively charged and a positively charged species. In some embodiments, the negative charged species is the polynucleotide and the positively charged specie is the casein nanoparticle.

In some embodiments, the plurality of particles of the invention has a spherical geometry or shape. In some embodiments, the plurality of particles has a spherical shape, a quasi-spherical shape, an elliptical shape, a quasi-elliptical sphere, a deflated shape, a rod shape, a concave shape, an irregular shape, or any combination thereof. One skilled in the art will appreciate that the exact shape of each of the plurality of particles may differ from one particle to another.

In some embodiments, at least 50, at least 70%, at least 80%, at least 90% by total weight of the casein nanoparticle constitutes of casein, including any salt thereof. In some embodiments, at least 70%, at least 80%, at least 90% by total weight of the casein nanoparticle constitutes of casein, including any salt thereof.

In some embodiments, the casein nanoparticles consist essentially of casein, including any salt thereof. In some embodiments, the casein nanoparticles are substantially devoid of a crosslinker. In some embodiments, the casein nanoparticles are non-crosslinked nanoparticles.

In some embodiments, a w/w ratio between the polynucleotide and the casein within the composition of the invention is between 0.1:1 and 2:1, between 0.1:1 and 1:1, between 0.3:1 and 1:1, between 0.3:1 and 2:1, including any range in between. A skilled artisan will appreciate that the ratio between the polynucleotide and the casein NP may be lower than 0.1:1, however, since the composition of the invention is predestined to carry the polynucleotides it is highly desirable to increase polynucleotide: casein NP ratio within the composition.

In some embodiments, the plurality of particles of the invention is characterized by a polydispersity index (PDI) ranging between 1.1 and 1.5, including any range in between.

In some embodiments, the casein nanoparticles comprise any one of: α Casein, α-s2 Casein, β-Casein, κ-Casein, or any combination thereof. In some embodiments, the casein nanoparticles are composed essentially of: α Casein, α-s2 Casein, β-Casein, or κ-Casein, or any combination thereof. In some embodiments, at least 70%, at least 80%, or between 70 and 99%, between 80 and 95% by dry weight of the casein nanoparticles consist of any one of: α-s1 Casein, α-s2 Casein, β-Casein, and κ-Casein, including any salt and any combination thereof. In some embodiments, the casein in the casein nanoparticles is a protein isolate. In some embodiments, the protein isolate is devoid of chemical modification. In some embodiments, the protein isolate is a milk isolate. In some embodiments, the protein isolate is a bovine milk isolate.

In some embodiments, the term “polynucleic acid” and the term “polynucleotide” are used herein interchangeably. In some embodiments, the polynucleotide comprises 60 to 15000 nucleobases, 15000 to 10000, 10000 to 4700, 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 20 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the plurality of particles of the invention comprises a plurality of nanoparticle types. In some embodiments, each particle comprises: (i) a different polynucleotide or (ii) a combination of one or more different polynucleotide.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, at least 1 nucleobase, at least 1 nucleobase, at least 1 nucleobase, at least 1 nucleobase, or at least 10 nucleobases, or any value and range therebetween. Each possibility represents a separate embodiment of the invention.

In some embodiments, a polynucleotide comprises RNA, DNA, a synthetic analog of RNA, a synthetic analog of DNA, DNA/RNA hybrid, or any combination thereof. In some embodiments, the plurality of nanoparticles of the invention comprises a polynucleotide selected from: RNA, DNA, a synthetic analog of RNA, a synthetic analog of DNA, DNA/RNA hybrid, or any combination thereof.

In some embodiments, the polynucleotide comprises an inhibitory nucleic acid. In some embodiments, the polynucleotide comprises an antisense oligonucleotide.

As used herein, an “antisense oligonucleotide” refers to a nucleic acid sequence that is reversed and complementary to a DNA or RNA sequence.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments, the inhibitory nucleic acid has one or more chemical modifications to the backbone or side chains. In some embodiments, the inhibitory nucleic acid has at least one locked nucleotide, and/or has a phosphorothioate backbone.

Non-limiting examples of inhibitory nucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)-of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the RNAi is biopesticide. As used herein the term “biopesticide” refer to natural compounds used in agricultural for treating pests and pathogens.

Non-limiting examples of pests include but are not limited to, insects, mites, ticks (and other arthropods), mice, rats, and other rodents, slugs, snails, nematodes, cestodes (and other parasites), weeds, fungi, bacteria, viruses and other pathogens.

In some embodiments, the pathogen is selected from: a virus, a bacterium, a fungus, a protozoan (such as but not limited to zoosporic protozoa), a nematode, or an arthropod. As used herein, the term “pathogen” and “pest” are interchangeable.

In some embodiments, the pathogen is a virus. In some embodiments, the pathogen is an arthropod. In some embodiments, the pathogen is a nematode. In some embodiments, the pathogen is a protozoan. In some embodiments, the virus is transmitted via any one of: arthropod, a nematode, a protozoan.

In some embodiments, the arthropod comprises an insect or an arachnid, including any developmental stage thereof, e.g., larvae, nymph, etc.

In some embodiments, the virus comprises a virus being transmitted by the obscure or tubber mealybug (e.g., Pseudococcus viburni)

Plant pathogens and/or pest are common and would be apparent to one of ordinary skill in the art.

In some embodiments, the polynucleotide comprises an expression vector or plasmid. In some embodiments, a vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

In some embodiments, the polynucleotide encodes a member of a clustered regularly interspaced short palindromic repeat (CRISPR) class I or class II system. In some embodiments, a member of the CRISPR system comprises a Cas protein, a single guide RNA (sgRNA), or both.

As used herein, “CRISPR” or “CRISP arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some embodiments, the spacers comprise 20 to 35 nucleotides. In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, CRISPR comprises CRISPR Class 2 system. In some class 2 system comprises CRISPR type II system.

The term “CRISPR type II” system refers to a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein. CRISPR-Cas systems fall into two classes. Class 1 systems use a complex of multiple Cas proteins to degrade foreign nucleic acids. Class 2 systems use a single large Cas protein for the same purpose. More specifically, class 1 may be divided into types I, III, and IV and class 2 may be divided into types II, V, and VI.

The type II CRISPR-Cas systems include the ‘HNH’-type system (Streptococcus-like; also known as the Nmeni subtype, for Neisseria meningitidis serogroup A str. Z2491, or CASS4), in which Cas9 is sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the polynucleotide comprising a nucleic acid sequence encoding at least one Cas gene comprises at least one Cas gene of type II CRISPR system (either typeII-A or typeII-B). In some embodiments, the at least one Cas gene of type II CRISPR system comprises a Cas9 gene. It should be appreciated that such system may further comprise at least one of Cas1, Cas2, csn2 and Cas4 genes.

In some embodiments, a Cas protein consists or comprises a Cas9 protein. Double-stranded DNA (dsDNA) cleavage by Cas9 is a hallmark of “type II CRISPR-Cas” immune systems. The CRISPR-associated protein Cas9 is an RNA-guided DNA endonuclease that uses RNA: DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosome Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

In some embodiments, the vector is a DNA plasmid delivered via non-viral methods or via viral methods. In some embodiments, the viral vector is a retroviral vector, a herpesviral vector, an adenoviral vector, an adeno-associated viral vector or a poxviral vector. In some embodiments, the promoters are active in mammalian cells. In some embodiments, the promoters are viral promoters.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, the composition of the invention further comprises an additive (e.g., colorant, UV blocker, stabilizer, antioxidant, preservative, etc.).

In some embodiments, the plurality of the nanoparticles of the invention is referred to as stable, if at least 80%, at least 85%, at least 90%, at least 95%, at least 99%, by weight of the particles retain at least 90% of the particle size, including any range therebetween.

In some embodiments, the plurality of nanoparticles of the invention is referred to as stable, when at least 80%, at least 85%, at least 90%, at least 95%, at least 99%, by weight of the particles remain at least 90% of the particle size within a solution (e.g. an aqueous solution) for a period of at least 1 h, at least 3 h, at least 5 h, at least 10 h, at least 24 h, at least 2 d, at least 10 d, at least 20 d, at least 1 m, at least 6 m, at least 1 year, including any value and range therebetween. Each possibility represents a separate embodiment of the invention.

In some embodiments, the plurality of the nanoparticles of the invention is referred to as stable by weight of the particles remain between 80 and 99%, between 80 and 85%, between 85 and 90%, between 90 and 95%, between 95 and 99%, including any range in between, of the particle size within a solution.

In some embodiments, the plurality of the nanoparticles of the invention is referred to as stable, when at least 80%, at least 85%, at least 90%, at least 95%, at least 99%, by weight of the particles retain at least 80% of the initial polynucleotide content. In some embodiments, the plurality of the nanoparticles of the invention is referred to as stable by weight of the particles retain between 80 and 95%, between 80 and 85%, between 85 and 90%, between 90 and 95%, including any range in between, of the initial polynucleotide content.

In some embodiments, the composition of the invention is an agricultural composition, comprising the plurality of nanoparticles of the invention and an agriculturally acceptable carrier. In some embodiments, the carrier is an agriculturally acceptable carrier. In some embodiments, an agriculturally acceptable carrier comprises an environmentally acceptable carrier. Such carriers can be any material that an animal, a plant or the environment to be treated can tolerate. In some embodiments, the carrier comprises any material, which can be added to the plurality of nanoparticles of the invention, or a composition comprising same, without causing or having an adverse effect on the environment, or any species or an organism other than the pathogen. Furthermore, the carrier must be such that the nanoparticle or composition comprising same, remains effective for introducing a polynucleotide to a plant and/or preventing or treating a viral infectious disease in a plant.

In some embodiments, the agricultural composition of the invention is formulated for delivery of the polynucleotide via foliage. In some embodiments, the agricultural composition of the invention is formulated for spraying, fogging, aerosol, or any other method compatible with foliage delivery. In some embodiments, the agricultural composition is a pesticide. In some embodiments, the agricultural composition is for use in a treatment of a plant disease. In some embodiments, the pesticide comprises a pesticidal effective polynucleotide sequence (i.e. a sequence complementary to at least one RNA/DNA sequence of the pest, and is capable for controlling the pest).

In some embodiments, the pest is a plant pest (e.g. a plant pathogen). In some embodiments, the plant pest comprises any one of: insects, mites, ticks (and other arthropods), mice, rats, and other rodents, slugs, snails, nematodes, cestodes (and other parasites), weeds, fungi, bacteria, viruses and other pathogens, including any combination thereof.

In some embodiments, the pathogen is selected from: a virus, a bacterium, a fungus, a protozoan (such as but not limited to zoosporic protozoa), a nematode, or an arthropod.

As used herein, the term “pathogen” and “pest” are interchangeable.

In some embodiments, the pathogen is a virus. In some embodiments, the pathogen is an arthropod. In some embodiments, the pathogen is a nematode. In some embodiments, the pathogen is a protozoan. In some embodiments, the virus is transmitted via any one of: arthropod, a nematode, a protozoan.

In some embodiments, the arthropod comprises an insect or an arachnid, including any developmental stage thereof, e.g., larvae, nymph, etc.

Plant pathogens and/or pest are common and would be apparent to one of ordinary skill in the art. In some embodiments, the virus comprises a genome comprising DNA, RNA, or a hybrid thereof. In some embodiments, the virus comprises a single stranded genome (e.g., the genomic matter, is made of a single stranded nucleic acid molecule). In some embodiments, the virus comprises a double stranded genome (e.g., the genomic matter, is made of two antiparallel nucleic acid molecules hybridized to one another).

In some embodiments, a concentration of the plurality of particles within the composition is between 0.1 and 100 mg/ml, between 0.1 and 10 mg/ml, between 1 and 100 mg/ml between 1 and 10 mg/ml, including any range between.

In some embodiments, the agriculturally acceptable carrier is selected from a group of: a solvent, a surfactant, a dispersant, a sticking agent, a spreading agent, a synergist, a penetrant, a compatibility agent, a buffer, a defoaming agent, a thickener, a drift retardant, or any combination thereof.

In some embodiments, the w/w concentration of the agriculturally acceptable carrier within the composition is between 0.1 and 99%, between 0.1 and 1%, between 1 and 10%, between 10 and 20%, between 20 and 30%, between 30 and 50%, between 50 and 60%, between 60 and 80%, or between 80 and 90%, including any range between. Each possibility represents a separate embodiment of the invention.

In some embodiments, the composition is formulated for administering by spraying, drenching, dipping, soaking, injecting, or any combination thereof. In some embodiments, the composition of the invention is formulated for administration by spraying. In some embodiments, the composition of the invention is formulated for administration as a spray or an aerosol.

### Methods

According to some embodiments, there is provided a method for manufacturing the plurality of nanoparticles of the invention, where in the method comprises (i) obtaining casein nanoparticles characterized by a positive average zeta potential, and (ii) contacting the casein nanoparticles with the polynucleotide at appropriate conditions, thereby forming the plurality of nanoparticles of the invention.

In some embodiments, the positively charged casein NPs of step (i) are characterized by a positive average zeta potential of below 23 mV, below 20 mV, below 18 mV, or between +5 and +20 mV, between +7 and +20 mV, between +10 and +20 mV, between +10 and +18 mV, between +10 and +17 mV, between +5 and +15 mV, between +7 and +15 mV, between +10 and +15 mV, between +5 and +30 mV, between +5 and +20 mV, between +10 and +30 mV, between +10 and +20 mV, including any range between, when measured at a pH value of about 4.5.

In some embodiments, step (i) comprises adding a salt (e.g. KCl, or NaCl) to a casein solution at a concentration suitable for formation of casein NPs. In some embodiments, formation of casein NPs is by casein coagulation. In some embodiments, a concentration suitable for formation of casein NPs is between 40 and 100 mM, between 45 and 50 mM, between about 50 and about 100 mM, or about 50 mM of the salt in the casein solution. In some embodiments, the casein NPs obtained by casein coagulation are negatively charged casein NPs. In some embodiments, the negatively charged casein NPs are characterized by an average zeta potential between −1 and −10 mV, or about-5, when measured at a pH of about 7.

In some embodiments, step (i) further comprising modifying an average zeta potential of the negatively charged casein NPs by exposing the negatively charged casein NPs to an acidic buffer solution, to obtain the positively charged casein NPs. In some embodiments, the acidic buffer solution has a pH of between 3 and 5, between 3.5 and 5, between 3.5 and 4.5, or about 4.5. In some embodiments, the acidic buffer solution is a MES buffer solution having a pH of about 4.5.

In some embodiments, appropriate conditions of step (ii) comprise a pH between 4 and 6, between 4 and 6, between 4 and 5, between 4.2 and 4.6, between 5 and 6, including any range between. In some embodiments, the method of the invention is performed by dispersing the casein NP in a buffer at a pH between 4 and 6, between 4 and 6, between 4 and 5, between 4.2 and 4.6, between 5 and 6, including any range between, to obtain positively charged casein NPs; and contacting the positively charged casein NPs with an appropriate amount of the polynucleotide. In some embodiments, the contacting step is performed at a pH between 4 and 7, between 5 and 6, between 4 and 5, between 4.2 and 4.6, between 5 and 7, between 6 and 7 including any range between.

In some embodiments, the appropriate conditions comprises a temperature between 5 and 40° C., between 5 and 30° C., between 1° and 40° C., between 1° and 30° C., between 2° and 40° C., between 2° and 30° C., including any range between; and a mixing period of time required to obtain the plurality of nanoparticles of the invention (e.g. between 10 min and 10 hours, including any range between). In some embodiments, contacting is performed under a pH between 4 and 7, between 4 and 6, including any range in between.

In some embodiments, contacting comprises mixing the casein nanoparticle and the polynucleotide at a casein NP to polynucleotide ratio between 1:0.1 and 1:2, between 1:0.1 and 1:1.5, between 1:0.1 and 1:1, between 1:0.2 and 1:1.5, between 1:0.3 and 1:2, between about 1:0.3 and about 1:1, including any range in between.

In some embodiments, the positively charged casein nanoparticles substantially (i.e. a particle size deviation of up to 10%) retain the average particle size of the negatively charged casein nanoparticles. In some embodiments, the particle size is measured by DLS (e.g. intensity based).

In some embodiments, the casein nanoparticles are characterized by a bi-modal particle size distribution. In some embodiments, the casein nanoparticles are characterized by a mono-modal particle size distribution. In some embodiment, the bi-modal particle size distribution comprises by a first average particle size distribution and a second average particle size distribution. In some embodiments, the first particle size distribution is characterized by an average particle size ranging between 10 and 30 nm, between 15 and 20 nm, between 20 and 25 nm, between 19 and 21 nm, between 15 and 30 nm, including any range in between. In some embodiments, the first average particle size distribution is 20 nm. In some embodiments, a percentage of particles characterized by the first average particle size distribution is between 5 and 15%, between 5 and 7%, between 7 and 9%, between 9 and 11%, between 11 and 13%, and between 13 and 15%, including any range in between.

In some embodiments, the second average particle size distribution is characterized by an average particle size distribution ranging between 100 and 200 nm, between 100 and 140 nm, between 130 and 140 nm, between 138 and 141 nm, between 130 and 180 nm, including any range in between. In some embodiments, the second average particle size distribution has a mean size of 139 nm. In some embodiments, a percentage of particles characterized by the second average particle size distribution is between 75 and 95%, between 75 and 80%, between 80 and 85%, between 85 and 90%, between 90 and 95%, and between 80 and 90%, including any range in between.

In some embodiments, the casein particles are characterized by a PDI ranging between 1.0 and 2, between 1 and 1.15, between 1.1 and 1.2, between 1 and 1.2, between 1.1 and 1.4, between 1.1 and 1.6, between 1.5 and 1.8, including any range in between.

According to some embodiments, there is provided a method for introducing a polynucleotide into at least one cell of a plant, the method comprises contacting the plant or a part of the plant, or an area under cultivation with an effective amount of the composition of the invention, thereby introducing a polynucleotide to the plant or plant part.

The term “effective amount” refers to a dosage of the nanoparticles of the invention applied to a plant or to an area under cultivation, sufficient to induce a biological response. In some embodiments, the biological response encompasses modification of at least one gene expression (e.g. inhibition, upregulation, or induction of gene expression) within a plant treated by the composition of the invention.

### Pest Control

Agricultural pests cause major yield and economic losses worldwide. Pests can develop resistance to chemical pesticides and breeding strategies faster than can be engineered for, hence there is an urgent need for alternatives in pest management strategies.

Development of RNAi biopesticides delivered by a nanoparticle carrier, which in turn is up-taken and delivered to the pest by the plant, could give an alternative to broad-spectrum chemical-based control measures for pests and pathogens, which would instead be targeted accurately and specifically with minimal off-target effects. Rapidly changing pathogens such as fungi, bacteria, and viruses, could be quickly characterized, sequenced, and included in an RNAi biopesticide—a clear advantage over standard pest control practices today which take a few years to breed resistance or engineer chemical protections for.

According to some embodiments, the herein disclosed nanoparticle and method of using same, are directed to pathogen or pest control. In some embodiments, the pathogen is a plant pathogen. According to some embodiments, there is provided a method for preventing or treating a viral infectious disease in a plant.

In some embodiments, the contacting of the composition of the invention to the plant or plant part is by, spraying, drenching, dipping, soaking, injecting, or any combination thereof.

In some embodiments, preventing or treating comprises reducing: a titer of a virus in the circulation of a plant or a part thereof, in a cell of a plant, or a combination thereof, the number of viral particles in a the circulation of a plant or a part thereof, in a cell of a plant, or a combination thereof, the number and/or stability of RNA molecules encoding a viral peptide, such as, but not limited to RdRp, CP, or both, the expression levels of RNA molecules encoding a viral peptide, such as, but not limited to RdRp, CP, or both, in the circulation of a plant or a part thereof, in a cell of a plant, or a combination thereof, the amount of a viral peptide, such as, but not limited to RdRp, CP, or both, in the circulation of a plant or a part thereof, in a cell of a plant, or a combination thereof. In some embodiments, preventing or treating comprises reducing pathogen loading on or within the plant or a part thereof, in a cell of a plant, or a combination thereof.

In some embodiments, preventing or treating comprises reducing the survival of a pathogen. In some embodiments, preventing or treating comprises reducing the replication rate of a pathogen. In some embodiments, preventing or treating comprises reducing the tolerability of a pathogen to standard therapy and/or prophylactics. In some embodiments, preventing or treating comprises increasing the susceptibility and/or vulnerability of a pathogen to standard therapy and/or prophylactics.

In some embodiments, preventing or treating comprises reducing: number of curled leaves of a plant, rate of downward curling or cupping of leaves of a plant, or any combination thereof.

In some embodiments, contacting comprises applying the agricultural composition (e.g. by spraying, fogging, etc.) to the plant or a part thereof. In some embodiments, contacting comprises applying the agricultural composition in a vicinity of a plant or a part thereof. In some embodiments, contacting comprises applying the agricultural composition to a growth medium comprising a plant. In some embodiments, the growth medium comprise soil.

In some embodiments, vicinity is at a distance of 10 cm to 50 cm, 1 cm to 100 cm, 10 cm to 1 m, 0.5 m to 2.5 m, 1 m to 50 m, 0.1 m to 30 m. each possibility represents a separate embodiment of the invention.

In some embodiments, a plant part comprises at least one leaf of the plant. In some embodiments, a plant part comprises one or more leaves of the plant. In some embodiments, a plant part comprises at least a portion of the foliage of the plant. In some embodiments, a plant part comprises the foliage of the plant.

As used herein, the terms “treatment” or “treating” of a disease, disorder or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, alleviated symptoms of the disease, disorder or condition.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

In some embodiments, ameliorating comprises alleviating at least one symptom associated with a disease as described herein.

### GENERAL

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “consisting essentially of” means that the composition, may include additional ingredients (e.g. in an amount of between 0.1 and 20% by dry weight of the composition) besides the casein and/or salt thereof and the polynucleotide (e.g. coloring agents, stabilizer, a salt, thickening agent, preservative, etc.) but only if the additional ingredients do not materially alter stability, average particle size, plant delivery, and/or biological function of the claimed composition.

As used herein, the term “about” when combined with a value refers to plus and minus 10% of the reference value (±10%). For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials

Pristine CNP particles were prepared from casein sodium salt from bovine milk (Sigma-Aldrich) and calcium chloride dihydrate (Merck). 6-aminofluorescein (6-AF) bioreagent, suitable for fluorescence, −95%, HPLC, 2-(N-morpholino) ethanesulfonic acid hydrate (MES, ≥95% titration), N-(3-dimethylaminopropyl)-N′-ethylcarbodiimide hydrochloride (EDC, commercial grade, ≥98%, HPLC). Double distilled water (Ultrapure water device, synergy-UV Type1, Treion), hydrochloric acid 37% (CARLO ERBA). Bisbenzimide H 33 342 trihydrochloride B2261.20 mg mL-1 (Merck), ethanol absolute (Merck).

### Example 1

### Preparation and Characterization of DNA-Protein Nanoparticles (PNs)

### Synthesis of Casein Nanoparticles (CNPs) (−7.5 mV)

1 gram of sodium caseinate was dissolved in 75 ml of double distilled water with stirring (600 rpm) for 20 min at room temperature to form homogenous solution. Then, 40 mL of calcium chloride solution (0.8% w/v) were added drop-wise with continuous stirring for 30 min at 600 rpm to obtain nanoparticles. After the formation of casein nanoparticles (CNPs), the solution was centrifuged at 10,000 rpm for 15 min, at least 5 times. The final supernatant was collected carefully and stored at 4° C. for further analysis.

Preparation of CNP Aqueous Dispersion at a Zeta Potential of −5.5+/−4.4 mV: One thousand milligrams of the resulting pellet was dispersed in 50 mL distilled water (pH 7) by ultrasonic finger (Sonics VibraCell 750 W, 35% amplitude) for 10 min and stored at 4° C. The obtained CNPs had a zeta potential of −5.5+/−4.4 mV.

Preparation of a Fluorescently Labeled CNP Aqueous Dispersion at a Zeta Potential of −1.7+/−4.1 mV: One thousand milligrams of the pristine CNP pellet was dissolved in 1 mL of MES buffer (pH 4.5, 0.1 m) to form a CNP dispersion in MES. Next, 2.4 mL of 0.5 mg mL-1 6-AF solution in an MES buffer (pH 4.5, 0.1 m) was added to the CNP dispersion, and the resulting CNP+6-AF mixture was sonicated by ultrasonic finger (Sonics VibraCell 750W, 35% amplitude) for 1 min to obtain a uniform dispersion. Five hundred microliters of 10 mg mL-1 EDC aqueous solution (double distilled water), as a zero-length cross-linker, was slowly added to the CNP+6-AF mixture. The resulting CNP+6-AF+EDC mixture was reacted for 6 h in a mixer (ELMI RM-2L Intelli-mixer) in the fine agitation mode. The obtained CNPs/6-AF conjugate aqueous dispersion was centrifuged at 10000 RPM for 10 min three times in an MES buffer (0.1 m, pH 4.5) to remove the unreacted reagents. The pellet was collected and redispersed in 50 mL of double distilled water (pH 7) by ultrasonic finger (Sonics VibraCell 750 W, 35% amplitude) for 10 min and stored at 4° C. The obtained CNPs had a zeta potential of −1.7+/−4.1 mV.

Preparation of Fluorescently Labeled CNP Aqueous Dispersion at a Zeta Potential of +13.0+/−4.9 mV: One thousand milligrams of the pristine CNP pellet was dissolved in 1 mL of MES buffer (pH 4.5, 0.1 m) to form a CNP dispersion in MES. Next, 2.4 mL of 0.5 mg mL-1 6-AF solution in MES buffer (pH 4.5, 0.1 m) was added to the CNP dispersion, and the resulting CNP+6-AF mixture was sonicated by ultrasonic finger (Sonics VibraCell 750 W, 35% amplitude) for 1 min to form a uniform dispersion. Five hundred microliters of 10 mg mL-1 EDC aqueous solution (double distilled water), as a zero-length cross-linker, was slowly added to the CNP+6-AF mixture. The resulting CNP+6-AF+EDC mixture was reacted for 6 h in a mixer (ELMI RM-2L Intelli-mixer) at fine agitation mode. The obtained CNPs/6-AF conjugates aqueous dispersion was centrifuged at 10000 RPM for 10 min three times in MES buffer (0.1 m, pH 4.5) to remove the unreacted reagents. The pellet was collected and redispersed in 50 mL of MES

buffer (0.1 m, pH 4.5) by ultrasonic finger (Sonics Vibra Cell 750 W, 35% amplitude) for 10 min and was stored at 4° C. The obtained CNPs had a zeta potential of +13.0+/−4.9 mV.

Preparation of Fluorescently Labeled CNP Aqueous Dispersion at a Zeta Potential of +23.0+/−5.6 mV: One thousand milligrams of the pristine CNPs pellet was dissolved in 1 mL of MES buffer (pH 4.5, 0.1 m) to form a CNP dispersion in MES. Then, 2.4 mL of 0.5 mg mL-1 6-AF solution in the MES buffer (pH 4.5, 0.1 m) was added to the CNP dispersion, and the resulting CNP+6-AF mixture was sonicated by ultrasonic finger (Sonics VibraCell 750 W, 35% amplitude) for 1 min to form a uniform dispersion. Five hundred microliters of 10 mg mL-1 EDC aqueous solution (double distilled water), as a zero-length cross-linker, was slowly added to the CNP+6-AF mixture. The resulting CNP+6-AF+EDC mixture was reacted for 6 h in a mixer (ELMI RM-2L Intelli-mixer) in the fine agitation mode. The obtained CNPs/6-AF conjugate aqueous dispersion was centrifuged at 10000 RPM for 10 min three times in an MES buffer (0.1 m, pH 4.5) to remove the unreacted reagents. The pellet was collected and redispersed in 49.8 mL of MES buffer (0.1 m, pH 4.5)+200 μl 0.1 m HCL solution by ultrasonic finger (Sonics VibraCell 750W, 35% amplitude) for 10 min and stored at 4° C.

The resulting pH was 2.72, and the obtained CNPs had a zeta potential of +23.0+/−5.6 mV.

Preparation of DsRed Plasmid: Plasmid pSAT6-DsRed2-C1 AY818375 was used. Plasmid DNA was purified from E. coli with the Presto Mini Plasmid Kit (Geneaid, Cat. #PDH300), according to the manufacturer's protocol.

Preparation of the Different CNP Hoechst-DsRed Plasmid Electrostatic Conjugates: Hoechst (bisbenzimide) was used to label DsRed plasmid by a non-covalent reaction. A stock solution of Hoechst (20 mg mL-1) was prepared. DsRed plasmid (3000 ng μl-1) was diluted in 1 μl of MES buffer (0.1 m, pH 4.5) to obtain a concentration of 1500 ng μl-1 DsRed plasmid. Next, 5 μl of the Hoechst stock solution was added to 1500 ng μl-1 DsRed plasmid. The solution was gently mixed by slow pipetting to ensure proper distribution of Hoechst in the plasmid solution. The mixture was incubated for 30 min at room temperature in an ELMI RM-2L Intelli-mixer using the fine agitation mode. After the incubation, ethanol absolute was added to the DNA/Hoechst mixture to precipitate the DNA. The mixture was incubated at low temperature (−20° C.) for 12 h to enhance the precipitation process. Following the incubation, the mixture was centrifuged at 13500 RPM for 15 min to help DNA precipitation.

Then, 500 μl CNPs with a zeta potential of 13.0+/−4.9 mV (1000 mg of the pristine CNP pellet in 50 mL MES buffer) were added to the Hoechst/DNA mixture, gently mixed, and centrifuged again at 10000 RPM for 15 min to form a pellet containing CNPs/Hoechst/DNA plasmid conjugates. The supernatant was removed and the pellet was washed three times with cold ethanol to remove excess dye. After washing, the ethanol was removed to obtain a wet pellet. For pellet redispersion 1 mL of MES buffer (0.1 m, pH 4.5) was added to the CNPs/Hoechst/DNA plasmid pellet. The mixture was sonicated for 10 min at 20 AMPL, after which 1 mL of MES buffer (0.1 m, pH 4.5) containing 50 μl of ethanol was added, and the mixture was sonicated again for 10 min at 30 AMPL.

### Preparation of the Different CNP DsRed Plasmid Electrostatic Conjugates:

One milliliter of each dispersion was sonicated by ultrasonic multiprobe for redispersion of the CNPs (Sonics VibraCell 750 W, 35% amplitude) for 2 h. The dispersions were CNPs (pH 7, −5.5+/−4.4 mV), CNPs/6-AF (pH 7, −1.7+/−4.1 mV), CNPs/6-AF (pH 4.5, +13.0+/−4.9 mV), and CNPs/6-AF (pH 2.72, +23.0+/−5.6 mV). Ten microliters of a total DsRed plasmid solution of 180 ng μl-1 was gently added to 100 μl of each dispersion for 24 h electrostatic adsorption, and then diluted. Hundred microliters were added to 900 μl buffer (each CNP dispersion was diluted in its original buffer).

Plant and Growth Conditions: N. benthamiana plants were grown in soil in a light-controlled chamber at 25° C. with a 16 h photoperiod. Large, well-expanded leaves of five-week-old N. benthamiana plants were used for infiltration.

Infiltration of the CNP DsRed Plasmid Dispersions into N. benthamiana: The different CNP/DsRed plasmid electrostatic conjugates were infiltrated into N. benthamiana leaves. Five-week-old N. benthamiana plants were used in all the experiments. The CNP/DsRed conjugates were infiltrated immediately after the electrostatic adsorption into the lower epidermis of the half leaves. Infiltration of the CNPs 6-AF (pH 4.5, +13.0 mV) Aqueous Dispersion into N. benthamiana: One milliliter of 1000 mg mL-1 CNPs/6-AF (pH 4.5, +13.0+/−4.9 mV) was redispersed by ultrasonic multiprobe (Sonics Vibra Cell 750 W, 35% amplitude) for 2 h. The resulting dispersion (10, 20, 40, 100, and 300 μl) were diluted in an MES buffer (0.1 m, pH 4.5) to obtain 1 mL CNPs/6-AF (pH 4.5, +13.0+/−4.9 mV) dispersions at five different concentrations of 0.2, 0.4, 0.8, 2, and 6 mg mL-1, respectively. The resulting diluted dispersions were infiltrated into N. benthamiana leaves.

Five-week-old N. benthamiana plants were used in all the experiments. The dispersions were infiltrated immediately into the lower epidermis of the half leaves.

Infiltration of the CNPs 6-AF DsRed Plasmid (pH 4.5, +13.0+/−4.9 mV) Conjugate into N. benthamiana: One milliliters of 1000 mg mL-1 CNPs/6-AF (pH 4.5, +13.0+/−4.9 mV) was redispersed by ultrasonic multiprobe (Sonics VibraCell 750 W, 35% amplitude) for 2 h. Four different amounts of DsRed plasmid were added to 100 μl CNPs/6-AF/DsRed plasmid (pH 4.5, +13.0+/−4.9 mV) dispersion: 2, 6, 10, and 20 μg to form CNP dispersions with four different CNPs with DsRed plasmid weight ratios of 1:0.001, 1:0.003, 1:0.005, and 1:0.01 respectively. Hundred microliters of CNPs/6-AF/DsRed plasmid at each weight ratio went through 24 h. Electrostatic adsorption and then was diluted in 900 μl of MES buffer (0.1 m, pH 4.5) to form the optimal CNPs/6-AF concentration for internalization of 2 mg mL-1, in all ratios. The dispersions were infiltrated into N. Benthamiana leaves. Five-week-old N. benthamiana plants were used in all the experiments. The dispersions were infiltrated immediately into the lower epidermis of the half leaves.

Internalization and Successful Gene Expression versus Time of CNP 6-AF DsRed Plasmid (pH 4.5, +13.0+/−4.9 mV) Conjugates in N. Benthamiana cells: The internalization and successful gene expression versus time of CNPs/6-AF/DsRed plasmid (pH 4.5, +13.0+/−4.9 mV) conjugates in N. benthamiana cells was characterized by confocal fluorescence microscopy.

The internalization rate was monitored based on the green fluorescence emission of the 6-AF. The expression rate of the DsRed plasmid was based on the red fluorescence emission of the DsRed protein. To quantify the red and green fluorescent signals versus time, 100 μl of CNPs/6-AF/DsRed plasmid (pH 4.5, +13.0+/−4.9 mV) dispersion with CNPs was infiltrated into N. benthamiana leaves, the DsRed plasmid at a weight ratio of 1:0.01 to form an optimal CNPs/6-AF concentration for internalization of 2 mg mL-1.

Confocal Microscopy: Micrographs were acquired with a Leica SP8 laser scanning microscope (Leica, Wetzlar, Germany), equipped with a solid-state laser with 488 and 552 nm light, an HC PL APO CS 63×/1.2 water immersion objective (Leica, Wetzlar, Germany), and Leica Application Suite X software (LASX, Leica, Wetzlar, Germany).

Green (CNPs) had excitation of 488 nm emission range of 500-540 nm. Red (DsRed protein) had excitation of 552 nm and emission range of 565-640 nm. The emission signals were detected by hybrid (HyD) detectors.

A red auto-fluorescence emission signal (chloroplast) of 660-720 nm was detected with a PMT detector and converted to blue for convenience.

A series of optical sections through the depth of a sample (z-stack) within a defined volume were reconstructed into a 3D model to produce a highly accurate 3D reconstruction of the entire sample.

Cryogenic SEM Microscopy, Cryofixation, and Freeze-Fracture: Plant tissue samples were frozen between two aluminum discs and cryoimmobilized in a high-pressure freezing device (EM ICE, Leica); 20% PVP40 was used as a cryoprotectant. The frozen samples were then mounted on a holder under liquid nitrogen in a specialized loading station (EM VCM, Leica). They were transferred under cryogenic conditions (EM VCT500, Leica) to a sample preparation freeze fracture device (EM ACE900, Leica) and fractured at −120° C. Samples were etched for 3 min at −110° C. and coated with 3 nmPtC. Samples were imaged in an HRSEM Gemini 300 SEM (Zeiss) by a secondary electron in-lens detector while maintaining an operating temperature of −120° C.

Hydrodynamic Diameter and Zeta Potential Characterization by Dynamic Light Scattering (DIS): The hydrodynamic diameter and the zeta potential of the CNPs were characterized by MasterSizer 2000, Malvern Panalytical, Malvern (UK). Hundred microliters of each dispersion was diluted in 900 μl of the appropriate buffer. The dispersions were CNPs (pH 7, −5.5+/−4.4 mV), CNPs/6-AF (pH 7, −1.7+/−4.1 mV), CNPs/6-AF (pH 4.5, +13.0+/−4.9 mV), and CNPs/6-AF (pH 2.72, +23.0+/−5.6 mV). For the hydrodynamic diameter, each diluted sample was added to a particle size analytical sample DTS0012 cell. For the zeta potential measurements, each diluted sample was added to a zeta potential sample DTS1070 cell. The data were analyzed by Zetasizer Software (Version 7.11, Malvern Instruments Ltd., Malvern/UK).

Transmission Electron Microscopy: TEM at cryogenic temperature (Cryo-TEM) was used for direct imaging of the solutions and dispersions. Vitrified specimens were prepared on a copper grid coated with a perforated lacey carbon 300 mesh (Ted Pella Inc.). A typically 2.5 μl drop from the solution was applied to the grid and blotted with a filter paper to form a thin liquid film of solution. The blotted samples were immediately plunged into liquid ethane at its freezing point (−183° C.). The procedure was performed automatically in the plunger (Leica EM GP2). The vitrified specimens were then transferred into liquid nitrogen for storage. The samples were studied using a FEI Talos F200C TEM, at 200 kV, maintained at −180° C. Images were recorded on a FEI Ceta 16 m camera (4k×4k CMOS sensor) at low dose conditions, to minimize electron beam radiation damage. The measurements were done at the Ilse Katz Institute for Nanoscale Science and Technology, Ben-Gurion University of the Negev, Beer Sheva, Israel.

RNA Extraction and RT-PCR Analysis: Total RNA was extracted from 100 mg of Nicotiana benthamiana leaf tissue of the selected samples using the Hybrid-R RNA isolation kit (GeneAll, Seoul, South Korea) according to the manufacturer's protocol. The DNase and reverse-transcription reactions were performed on 1 μg of total RNA with the Maxima First-Strand cDNA Synthesis Kit (Thermo Scientific, Waltham, MA, USA) according to the manufacturer's instructions. The cDNA samples were diluted 1:10 (v/v) with ultrapure water and used as templates for subsequent reactions.

End-point PCR was used in order to amplify specific target genes. PCR amplicons were separated by agarose gel electrophoresis and visualized using the ENDURO GDS Gel Documentation System (Labnet International, Edison, NJ, USA).

Quantitative real-time PCR (qRT-PCR) was performed using Fast SYBR green Master Mix in a StepOnePlus Real-Time PCR System (Applied Biosystems, Waltham, MA, USA). The PCR conditions were as follows: 95° C. for 20 s, followed by 40 cycles of 95° C. for 3 s and 60° C. for 20 s. The samples were normalized using Nicotiana benthamiana actin as endogenous control and the relative expression levels were measured using the 2^{(-ΔΔCt)} analysis method. Results were analyzed with StepOne software v2.3.

Graphic Illustrations: The illustrations were prepared using the online BioRender tool (https://biorender.com/).

Statistical Analysis: The data were analyzed using JMP 16.0. Data were presented as mean+/−standard deviation. Significance was set at p≤0.05. The fluorescence intensity data were log-transformed before analysis to stabilize variances. For 6-AF, data for 1, 24, and 48 h were compared overall by one-way ANOVA. After significance was ascertained by the F-test, pairs of times were compared by two-sided contrast t-tests. For DsRed, data for 24 h were compared with 48 h by two-sided t-test.

In this study, the inventors demonstrated the use of CNPs as an effective tool for DNA delivery into intact mature plant cells. The formation of CNPs introduced a tremendous structural variation compared to the native protein but the sequence of the protein remained unchanged, preserving the original protein configuration. Nanoparticle formation was intended to prevent immediate recognition by plant mechanisms, which would lead to rapid protein degradation and prevent its recognition by the target cells as a foreign entity, overcoming the plant's protection mechanisms.

The zeta potential of the CNPs was tuned to meet the requirements of successful DNA delivery into N. benthamiana cells. The zeta potential of the CNPs was varied by altering the pH at three values: 2.7, 4.5, and 7.0. To determine their location in the plant, the CNPs were covalently modified with 6-AF green fluorescent dye prior to pH alteration. The immobilizations were performed by an EDC coupling agent. A plasmid harboring the sequence of the red fluorescent protein DsRed was absorbed to the surface of the CNPs/6-AF by electrostatic interaction and served as the model DNA in this study. As a control, the inventors had also prepared negatively charged CNPs by covalent immobilization of polyacrylic acid (data not shown).

In addition, the CNPs were covalently modified with the fluorescent dye 6-aminofluorescein (6AF), via 1-ethyl-3-(−3-dimethyl aminopropyl) carbodiimide hydrochloride (EDC)/N-hydroxysuccinimide (NHS) carbodiimide coupling agents.

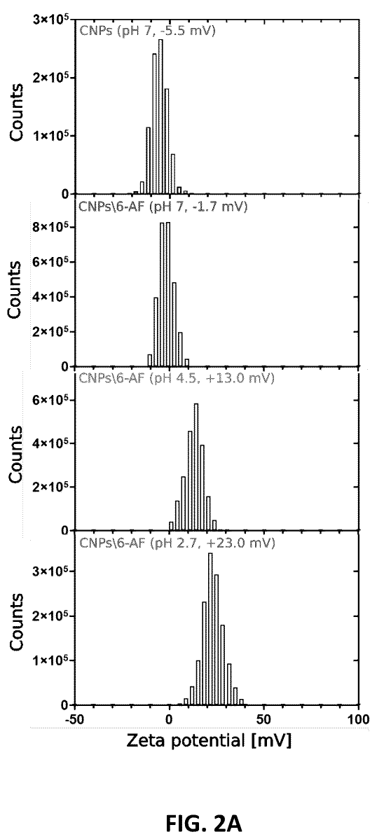

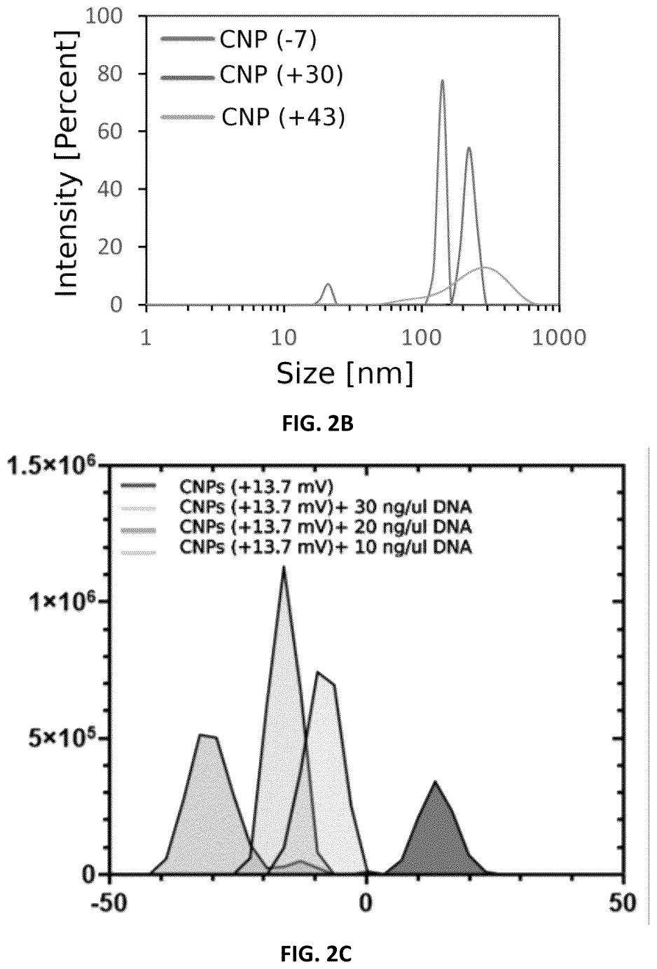

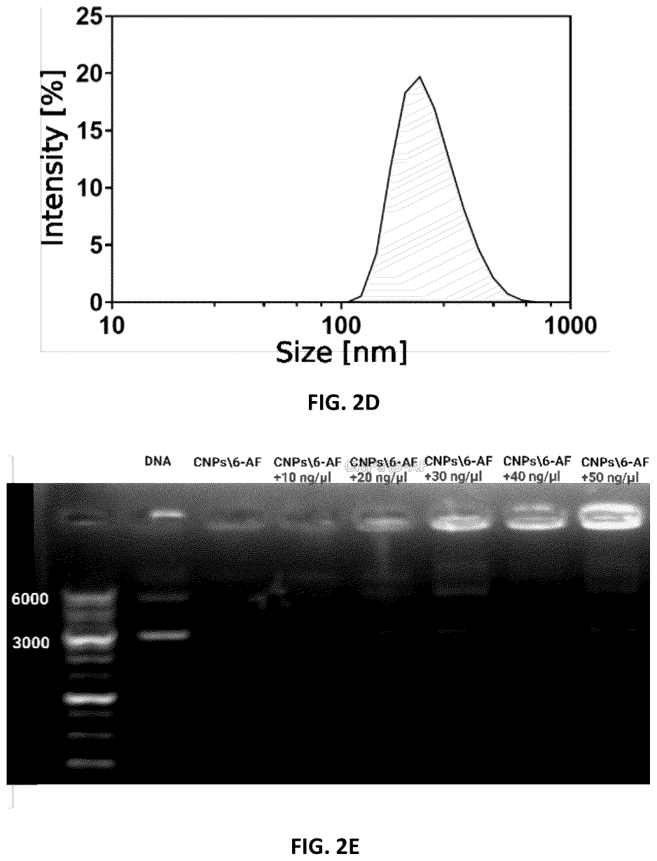

The zeta potential and the hydrodynamic diameter distributions of the CNPs at pH values of 7, 4.5, and 2.7 were characterized by DLS. Their values are shown in FIG. 2a,b, respectively.

The systems with their average hydrodynamic diameter and zeta potential averages were: CNPs (pH 7, −5.5±4.4 mV, 246±81 nm), CNPs/6-AF (pH 7, −1.7±4.1 mV, 318±40 nm), CNPs/6-AF (pH 4.5, +13.0±4.9 mV, 214±82 nm), and CNPs/6-AF (pH 2.7, +23.0±5.6 mV, 245±117 nm).

The zeta potential increased when the pH decreased. Introducing the CNPs/6-AF to pH 7 resulted in a negative average zeta potential of −5.5±4.4 mV, which is not sufficient for forming an electrostatic interaction with the negatively charged DNA. By contrast, introducing the CNPs/6-AF to pH values of 2.7 and 4.5 resulted in positive average zeta potential values of +23.0±5.6 and +13.0±4.9 mV, respectively, which should be sufficient to form an electrostatic interaction with DNA. Furthermore, the covalent immobilization of 6-AF also increased the zeta potential from −5.5±4.4 mV to −1.7±4.1 mV (at pH 7). This increase resulted from the amidation of the 6-AF through its amine groups with available carboxylic groups on the surface of the CNPs, reducing the number of carboxylic groups.

The hydrodynamic diameters of the two positive CNPs/6-AF with zeta potential values of +23.0±5.6 and +13.0±4.9 m V were 245±117 and 214±82 nm, respectively, and had a relatively high size distribution.

The ability of the different CNPs to deliver DNA to intact plant cells was investigated by their adsorption of DsRed plasmid. The expressed DsRed protein can be easily detected by confocal fluorescence microscopy because of its red fluorescence, therefore it served as an indicator of the efficiency of DNA delivery to the model plant, N. benthamiana. It is important to note that the fluorescent reagent, i.e., the DsRed plasmid itself is not fluorescent. However, the expressed DsRed protein is fluorescent, with an excitation maximum of 558 nm, and an emission maximum of 583 nm.

The studied systems, namely CNPs (pH 7, −5.5±4.4 mV, 246±81 nm), CNPs/6-AF (pH 7, −1.7±4.1 mV, 318±40 nm), CNPs/6-AF (pH4.5, +13.0±4.9 mV, 214±82 nm), and CNPs/6-AF (pH 2.7, +23.0±5.6 mV, 245 #117 nm) were adsorbed to the DsRed plasmid. The resulting electrostatic conjugates were infiltrated into the model plant N. benthamiana. The treated leaves were characterized by confocal microscopy to investigate the location of the CNPs/6-AF (which has a green fluorescent emission), and the expression rate of the DsRed plasmid by the red fluorescent emission of the expressed DsRed protein.

The results obtained by the inventors showed that the CNPs/6-AF/DsRed plasmid (pH 4.5, +13.0±4.9 mV, 214±82 nm) conjugates were characterized by superior expression of the DsRed protein and therefore were used in further experiments.

To gain an in-depth understanding of the structural aspects influencing the functionality of the CNPs/6-AF (pH 4.5, +13.0±4.9 mV, 214±82 nm), we used cryo-TEM for characterization. The cryogenic technique ensures that the structural features observed are representative of the nanoparticles in their native, biologically relevant state. This approach enables a detailed investigation of the CNP structure (FIG. 2c). The cryo-TEM results show that the CNPs have approximately spherical morphology, and the size distribution of the CNPs diameter that appears in the DLS data is also reflected in the cryo-TEM results. The results also show fluctuating nanoscale morphology attributed to molecular details of the casein itself.

The zeta potential of the CNPs/6-AF/DsRed plasmid (pH 4.5) conjugates was investigated at different CNPs: DsRed plasmid ratios (FIG. 2). At plasmid concentrations of 10, 20, and 30 ng μl-1, the corresponding average zeta potential values were +13.0±4.9, −8.57±1.2, −18.15±2.8, and −31.22±3.2 mV, respectively. These results confirm the electrostatic adsorption of the plasmid to the CNPs, and show a linear relationship with a good correlation of R2=0.9712 between the zeta potential of the conjugate and the plasmid concentration, indicating that pseudo first-order adsorption kinetics take place between the CNPs and the DsRed plasmid in these conditions.

In this study, we explored the potential of CNPs/6-AF (pH 4.5, +13.0±4.9 mV, 214±82 nm) as a carrier for different ratios of DsRed plasmid. An essential aspect was to ensure that CNPs retained their original size properties following plasmid absorption. To verify this, we measured the average hydrodynamic diameter of the CNPs/6-AF/DsRed plasmid conjugate at pH 4.5, using the highest plasmid concentration of 30 ng μl-1. The measurement yielded an average hydrodynamic diameter of 248 nm±16.6 nm, which remained virtually unchanged. The observation supports the individual absorbance between the CNPs and the plasmid (FIG. 2fd).

The minimal changes observed in the average diameter at the highest plasmid concentration attest to the dispersion and the stability of CNPs/6-AF (pH 4.5, +13.0±4.9 mV, 214±82 nm) as a carrier.

To make sure that CNPs/6-AF/DsRed plasmid conjugates remain stable and to conduct further examination of the concentration, allowing substantial DNA binding to the CNPs, the CNPs/6-AF/DsRed plasmid conjugates were characterized by ingel agarose electrophoresis (1%) assay.

This technique involves the application of an electric field, propelling DNA through the agarose gel from the negative to the positive electrode. CNPs/6-AF/DsRed plasmid conjugates are positively charged, which should keep them near the negative electrode during electrophoresis. The substantial size of the conjugates and the charge interaction with the gel hinder their mobility through the gel pores and effectively trap them in the well along with the DNA.

In this analysis, we examined CNPs/6-AF/DsRed plasmid conjugates (2 mgmL-1 CNPs/6-AF) at plasmid concentrations of 10, 20, and 30 ng μl-1 (as illustrated in FIG. 2d). We also introduced two additional DNA concentrations, 40 and 50 ng μl-1. As controls, we used CNPs and DsRed plasmid separately. As shown in FIG. 2g, the distinct DNA bands were observed at 3000 and 6000 base pairs in the control track. The CNPs could not be visualized and were not stained with ethidium bromide because this dye selectively labels DNA and cannot label the protein nanoparticles.

At the lowest DNA concentration of 10 ng μl-1, the DNA absorption was almost invisible, but as we increased the DNA concentration, a clear DNA band emerged within the gel well. This suggests a clear connection between increased DNA concentration and its affinity for CNPs.

### Example 2

### Internalization of CNPs/6-AF (pH 4.5, 13.0±4.9 mV) in N. benthamiana Cells

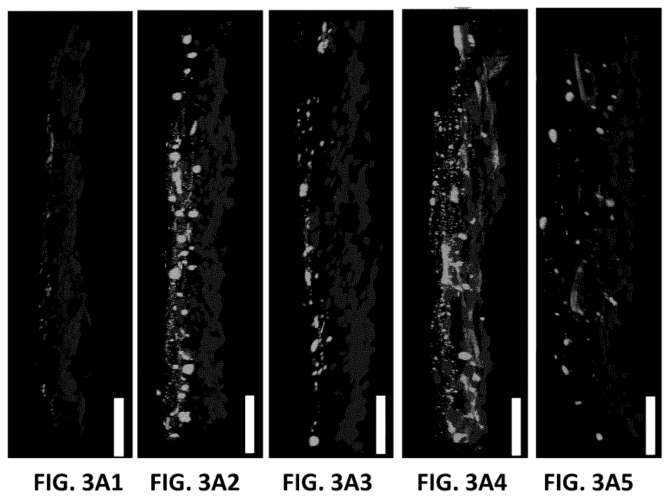

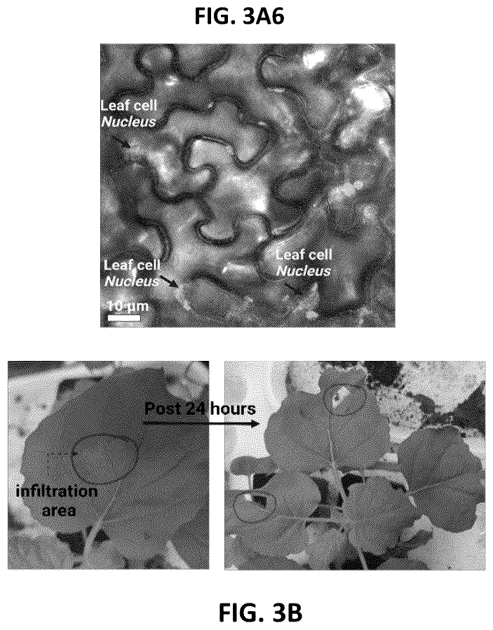

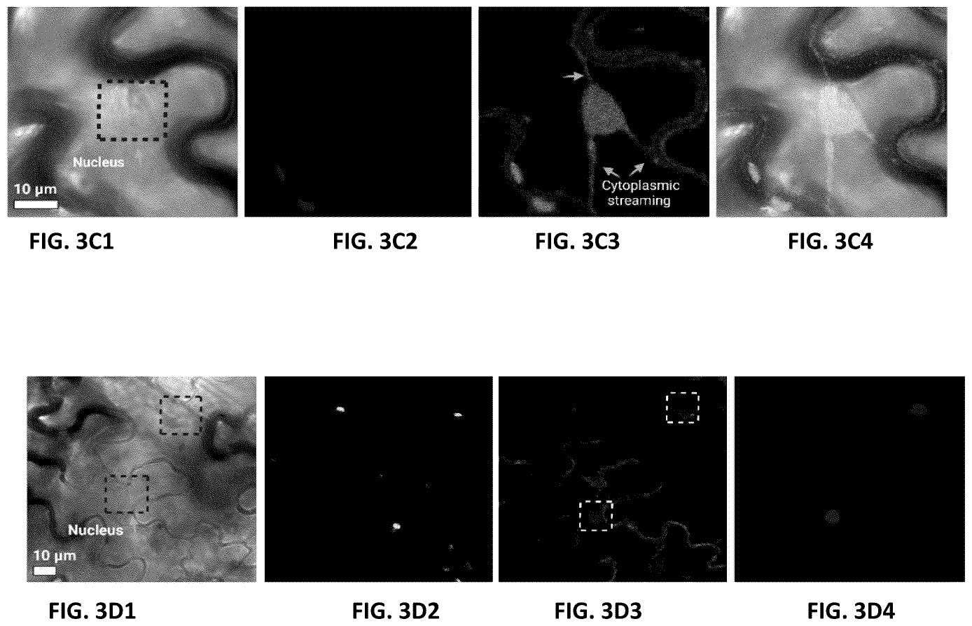

We investigated the intracellular and nucleus uptake of the CNPs/6-AF (pH 4.5, +13.0±4.9 mV, 214±82 nm) into N. benthamiana cells. To this end, CNPs/6-AF (pH 4.5, +13.0±4.9 mV, 214±82 nm) were infiltrated into the leaves of N. benthamiana and incubated for 2 h. The concentration of the CNPs in the infiltrated dispersion was varied to study the influence of the CNP concentration on their cellular internalization. Cross sectional analysis by confocal fluorescence microscopy showed the CNP locations throughout the observed cell layers. The internal part of the cells was detected by the red chloroplast autofluorescence, which was converted to blue for convenience. The results show that cellular internalization occurred only at a CNP concentration of 2 mg mL-1. By contrast, the CNPs remained outside and did not localize inside the N. benthamiana cells at any of the other concentrations. Inadequate particle quantities were observed outside the cells (0.2 to 0.8 mg mL-1 CNPs). In addition, an excessive concentration of particles (6 mg mL-1) may result in clustering outside the cells, hindering their entry into the cellular environment (FIG. 3a). The leaves (2 mg mL-1 CNPs) appeared viable (FIG. 3b—top).

The injected sections of the leaves (2 mg mL-1 CNPs) were further characterized by confocal fluorescence microscopy (FIG. 3b—bottom,c). FIG. 3b-bottom (low magnification) and c (high magnification) depicts the CNP localizations in the nucleus, cytoplasm, and chloroplast. The nucleus can be recognized by cytoplasmic streaming entering it and is also clearly seen in the 3D z-stack micrograph. In addition, to ensure nucleus identification, we applied Hoechst staining by injecting the Hoechst solution (20 mgmL-1) into the leaves. This staining process occurred ≈30 min before conducting the CLSM measurement; we maintained the same injection procedure for visualizing the particles (FIG. 3d). The results show colocalization of the green CNPs and the blue nucleus, as can be seen in the confocal micrographs (FIG. 3d).

The nanostructure of the N. benthamiana cells that were treated with CNPs/6-AF (pH 4.5, +13.0 mV, 214±82 nm) 1 h post-infiltration was characterized by cryo-SEM. To obtain high-resolution observation of the N. benthamiana cells at the nanoscale, we used the cryo-SEM technique enables to observe the samples in their near-native state. SEMmicrographs are obtained for ultrafast cooled vitrified cryo-SEM specimens prepared under controlled conditions (see materials and methods).

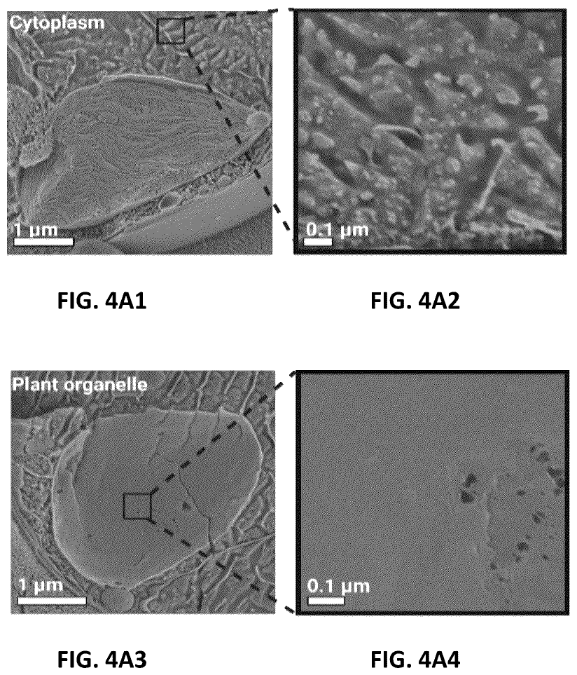

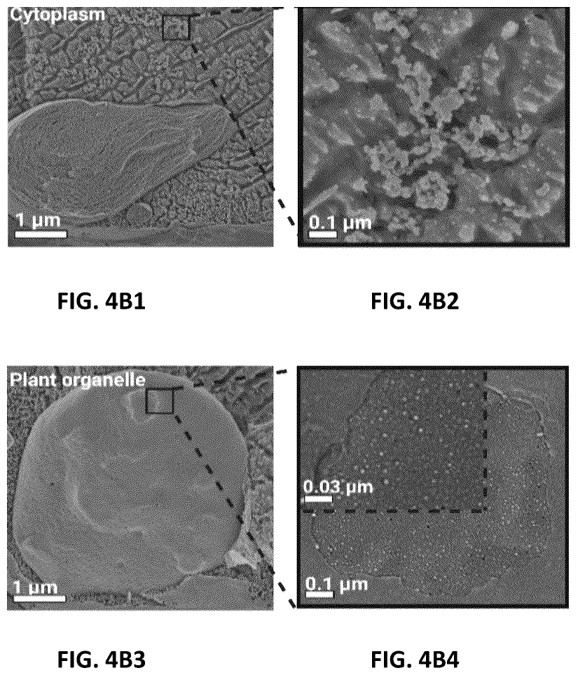

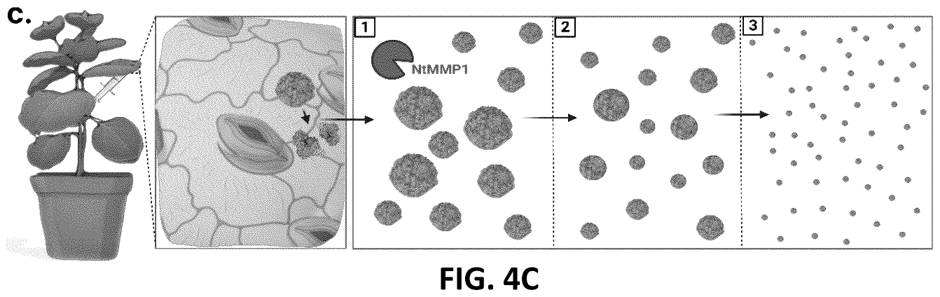

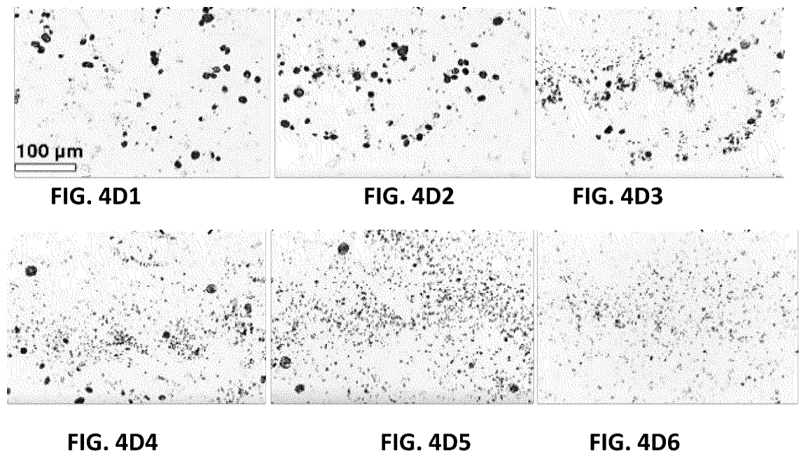

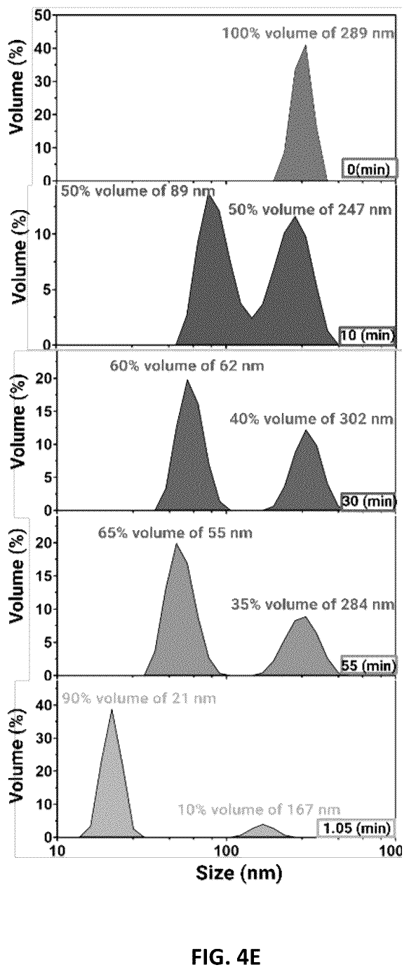

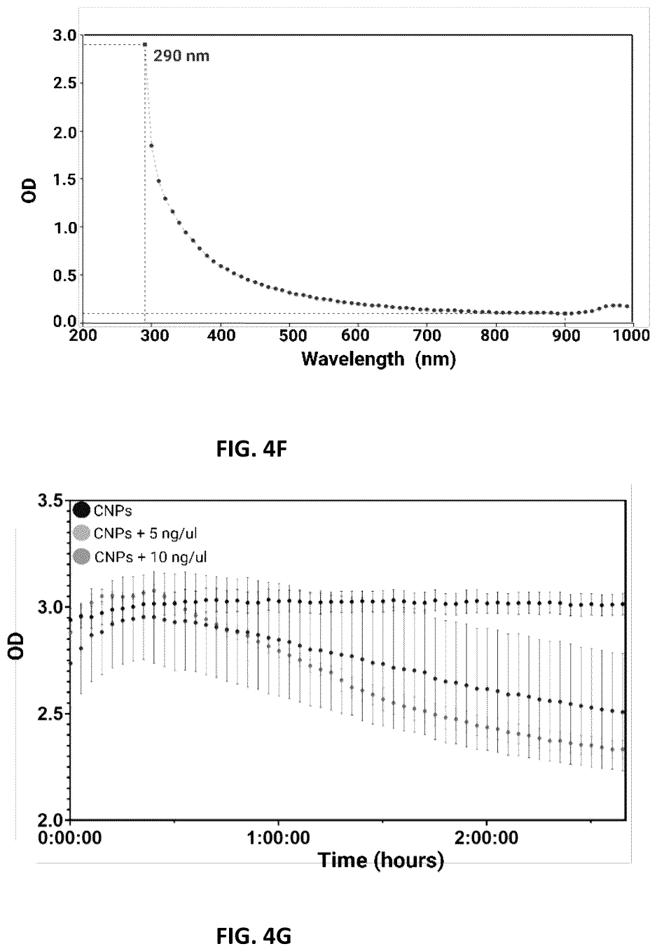

In this technique, the leaves are rapidly cooled, preserving their structure. The vitrified specimens were then fractured. Subsequently, the samples were captured under low-temperature conditions. The cryo-SEM characterization enabled us to obtain a detailed investigation of the location of the CNPs/6-AF (pH 4.5, +13.0 mV, 214±82 nm) in the N. benthamiana cells. The results are compared in FIG. 4a with non-treated plants (FIG. 4b).

The cryo-SEM micrographs show nanoscale granular structures that can be attributed to Golgi bodies in the treated and nontreated plants (FIG. 4a,b). The treated plants also accumulated colloidal particles with a diameter of 10 nm, which can be attributed to the CNPs. In addition, smaller particles (≈5 nm in diameter) were observed inside the organelles of the treated plants (FIG. 4b) but not in the non-treated plants (FIG. 4a). These results further support the successful internalization of the CNPs in the N. benthamiana cells and suggest that only a small fraction of the CNPs/6-AF (pH 4.5, +13.0 mV, 214±82 nm) with a relatively low diameter was internalized in the cells. Note, however, that even a small fraction of CNPs/6-AF (pH 4.5, +13.0 mV, 214 #82 nm) represents a considerably higher concentration than typically accepted for a noparticle infiltration into plants.

The accepted range of particle internalization is typically in the ng/ml cale because of concerns with the potential toxicity of the particles to the plant. Biodegradable CNPs, however, exhibit a remarkable ability to internalize within the plant cells at significantly higher concentrations (2 mg mL-1), demonstrating their unique potential for effective cargo delivery. These findings indicate that CNPs have the capacity to undergo degradation and enter plant cells through biological mechanisms connected to the plant. The dissimilarity in size between the synthesized CNPs/6-AF (average of 214±82 nm) and the CNPs/6-AF observed within the leaf has raised questions about the entry mechanism of these nanoparticles.

Casein is a dominant milk protein with physicochemical properties and an amphiphilic structure that supports its functionality in cargo carrier systems. Casein is a substrate of the first tobacco plant matrix metalloproteinase (NtMMP1). Plant extracellular matrix metalloproteinase (MMP) is a group of plant proteases that belong to a family of metalloendopeptidases acting on the plant extracellular matrix (ECM) and respond to stresses such as pathogen attacks. These enzymes have been isolated from various plant species, including Nicotiana tabacum.

NtMMP1 is enzymatically active as part of the plant's surveillance system for stress signals. Studies implementing casein zymography have shown that casein is a substrate of NtMMP1.

We hypothesized that the introduction of CNPs to the plant would result in a stress cascade that would activate the NtMMP1 and lead to some CNP degradation, which enable its internalization in the plant cell, as shown in FIG. 4c. We conducted a comprehensive analysis to ensure the degradability of our distinct CNPs/6-AF (pH 4.5, +13.0 mV, 214±82 nm). This investigation involved exposing CNPs to varying concentrations of matrix metalloproteinase-7 (MMP-7) and assessing the effect of degradation on CNPs using confocal microscopy, DLS analysis, and plate reader analysis, as illustrated in FIG. 4e-g.

We selected MMP-7 for this experiment because CNPs, the substrate of MMP-7, are also recognized by NtMMP1. Additionally, plant MMPs are phylogenetically close to human MMP-7. The enzymatic activity of NtMMP1 obeyed Michaelis-Menten kinetics. The computed kcat/Km value, determined to be 5.74×104±0.5×104 M-1 s-1, is similar to the kcat/Km value of the human MMP-7 for the same substrate. In Michaelis-Menten, the kcat/Km value serves as a metric for the catalytic efficiency of an enzyme. This value is obtained by dividing the turnover number (kcat, representing the number of substrate molecules converted to product per enzyme active site per unit time) by the Michaelis constant (Km, indicating the substrate concentration at which the reaction rate reaches half of Vmax). The similarity in kcat/Km values between NtMMP1 and human MMP-7 indicates a comparable catalytic efficiency, which suggests that both enzymes exhibit a similar capacity to convert substrate into product relative to substrate concentration.

To investigate the enzymatic activity of MMP-7 in terms of its ability to degrade the studied CNPs, we have qualitatively monitored the size of the CNPs that were applied on a glass slide together with the enzyme MMP-7 by optical microscopy. As shown in FIG. 4d, 0.5 μl of aggregated CNPs/6-AF (pH 4.5, +13.0 mV, 214±82 nm) were applied on a glass slide by dropping, followed by the addition of 0.5 μl of MMP-7 (0.1 mg mL-1). We used aggregated particles to highlight the degradable process, which is difficult to accomplish with particles alone. Over time, the aggregated CNPs/6-AF clusters underwent degradation into casein nanoparticles, noticeable from 5 to 40 min after the addition of MMP-7. The transformation of black dots to transparent ones between 30-40 min is a visual indicator of the advancing degradation process.

In addition to visualizing the degradation process through CLSM over time, we conducted further analysis by adding 200 μl MES buffer to 10 μl of dispersed CNPs/6-AF (2 mg mL-1) in a DLS cuvette. We used volume distribution in DLS analysis to measure the size distribution of particles in the suspension. This method is useful when the goal is to understand the overall volume of particles in the sample.

Before DLS analysis, we carried out a slow pipetting step to prevent the CNPs from aggregating at the bottom of the cuvette. In the absence of MMP-7, CNPs/6-AF exhibited a consistent size of 100% volume at 289±35 nm. After introducing 2 μl of MMP-7 (0.1 mg mL-1) to the dispersed CNPs/6-AF, we monitored changes in size.

At 10 min post-MMP-7 addition, two distinct peaks emerged, with 50% volume each at 89±20 and 247±60 nm, indicating the initiation of the degradation process. After 30 min, 40% of the particles exhibited a size of 302±50 nm, suggesting particle aggregation, while 60% of the volume remained at 62±10 nm, indicating ongoing particle degradation. At 55 min post-MMP-7 addition, the predominant volume shifted to smaller particles, with 65% at 55±10 and 35% at 284±60 nm. At 1.05 h after MMPs addition, 90% of the volume of particles was reduced to 21±3 nm and only 10% remained at 167±25 nm, showing significant reduction in the distinct CNPs/6-AF and attesting to the progression of the degradation process (FIG. 4e).

For the final analysis, we used a plate reader. We initially conducted absorbance spectra scanning of CNPs/6-AF but it did not reveal a distinct absorption wavelength because of interference from 6-AF, which was binding to the surface of the nanoparticles, affecting the absorbance of casein. To address this, we examined the absorbance spectra of CNPs without the green labeling, revealing a wavelength of 290 nm (FIG. 4f).

Subsequently, we added 100 μl of MES buffer (pH=4.5) to 100 μl of CNPs (2 mg mL-1) in each well. To the same amount of CNPs, we also added MMP-7 in two concentrations: 5 and 10 ng μl-1. We performed all three tests three times in different wells to obtain an average and standard deviation, as shown in FIG. 4g. The result indicates a constant absorbance of CNPs at the 290 nm wavelength, with a decrease in absorbance starting after 30 min with the addition of 5 ng μl-1 MMP-7. The decrease in absorbance was more pronounced when 10 ng μl-1 MMP-7 was added, starting at the 20 min mark and displaying a sharper decline. This observation suggests a concentration-dependent effect of MMP-7 on the degradation of CNPs, revealing dynamic changes over time.

We demonstrated the degradation of our unique CNPs/6-AF in the presence of MMP-7, mimicking the activity of NtMMP1. The degradation in the presence of MMP-7 is present whether CNPs are labeled or not.

Previous works have indicated that to obtain successful DNA delivery, the nanocarrier needs to meet the following three criteria: 1) The diameter of the nanocarrier must be smaller than the size exclusion limit (SEL) of the cell wall; 2) The DNA must be desorbed from the nanocarrier; and 3) For the specific case of protein-based nanovessel, it must be able to escape the endocytic pathway. We posited that the nanocarrier examined here, CNPs/6-AF/DsRed plasmid (pH 4.5, +13.0±4.9 mV, 214±82 nm), would meet these criteria because the diameter of the CNP/DNA conjugates can be reduced by the NtMMP1, which would facilitate its penetration through the cell wall and contribute to the detachment of the DNA from the CNPs. Studies have also indicated that histidine-containing peptides can escape the endocytic pathway and that NtMMP1 hydrolyzes CNPs through cleavage after histidine. This led to the hypothesis that the exposure of the CNPs/6-AF/DsRed plasmid (pH 4.5, +13.0±4.9 mV, 214±82 nm) would conjugate to the NtMMP1 and the histidine regions would be exposed, which would enhance their ability to escape the endocytic pathway.

This pioneering achievement in the field of protein nanoparticles demonstrated success in entering cells. The large surface area of the protein nanoparticles, compared to amino acids, helps load DNA more efficiently. The natural biodegradability of these nanoparticles also makes them suitable carriers for delivering DNA and points to a new promising way of improving targeted gene delivery in plant cells.

### Example 3

### Internalization of CNPs/Hoechst/DNA Plasmid (pH 4.5, 13.0±4.9 mV) to N. benthamiana Cells

This experiment was designed to explore the ability to obtain successful delivery f the model DNA DsRed plasmid interaction between DNA plasmid and the CNPs within the N. benthamiana cells. The dynamics of the DNA absorption onto the CNPs in the cells provides essential information about the ability of CNPs to safeguard the DNA from degradation and enable prediction of transcription and DsRed expression time within the plant system. To this end, we used the fluorescent dye Hoechst to label the DsRed plasmid.

A stock solution of Hoechst (20 mg mL-1) was prepared. DsRed plasmid (3000 ng μl-1) was diluted in MES buffer (0.1 m, pH 4.5) to obtain a concentration of 1500 ng μl-1 DsRed plasmid. Next, 5 μl of the Hoechst stock solution was added to 1500 ng μl-1 DsRed plasmid. The solution was gently mixed by slow pipetting to ensure proper distribution of Hoechst with the DNA plasmid.

The mixture was incubated for 30 min at room temperature, then at low temperature (−20° C.) for 12 h to enhance the precipitation process. Following the incubation, the mixture was centrifuged to help DNA precipitation, after which CNPs with a zeta potential of 13.0±4.9 mV were added to the Hoechst/DNA mixture and centrifuged again to form a pellet containing CNPs/Hoechst/DNA plasmid conjugates. For pellet redispersion, MES buffer (0.1 m, pH 4.5) was added to the CNPs/Hoechst/DNA plasmid pellet, and the mixture was sonicated.

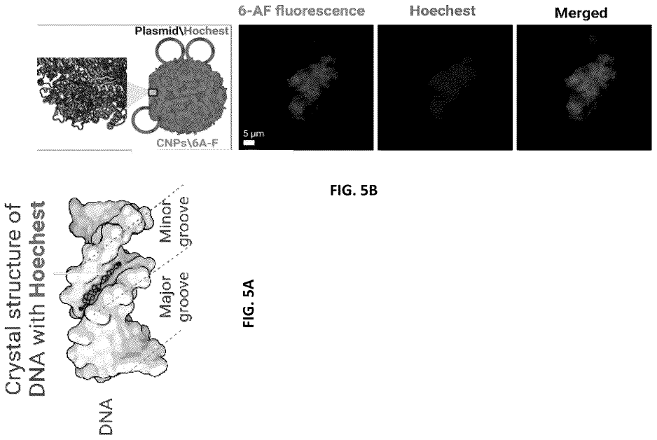

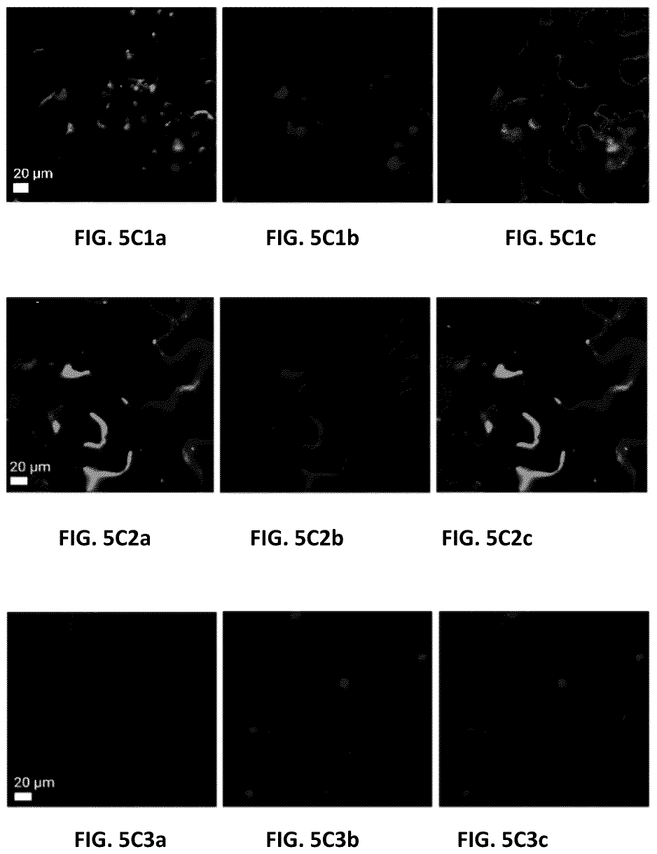

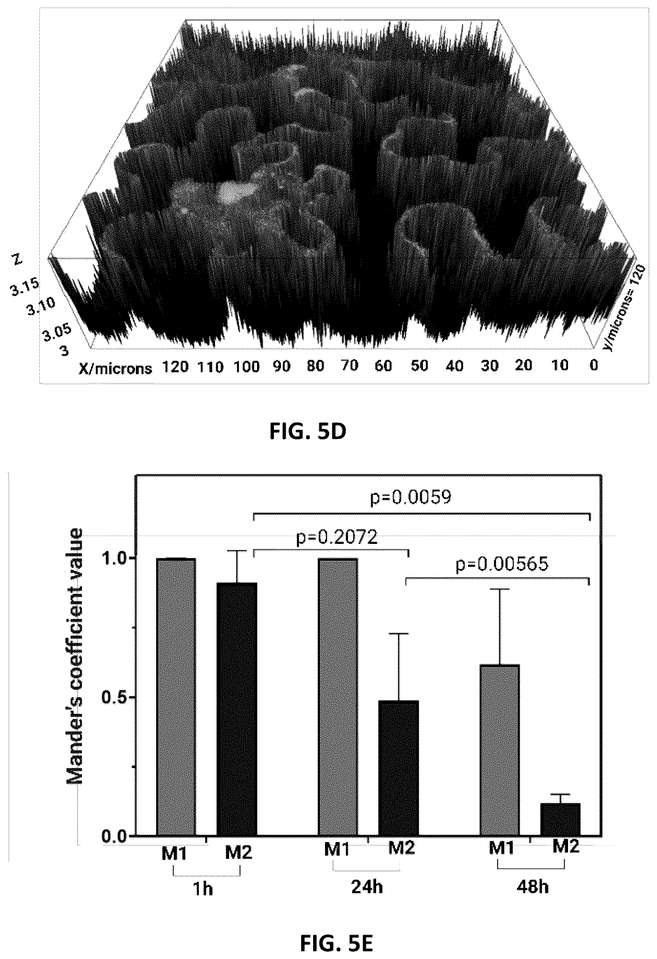

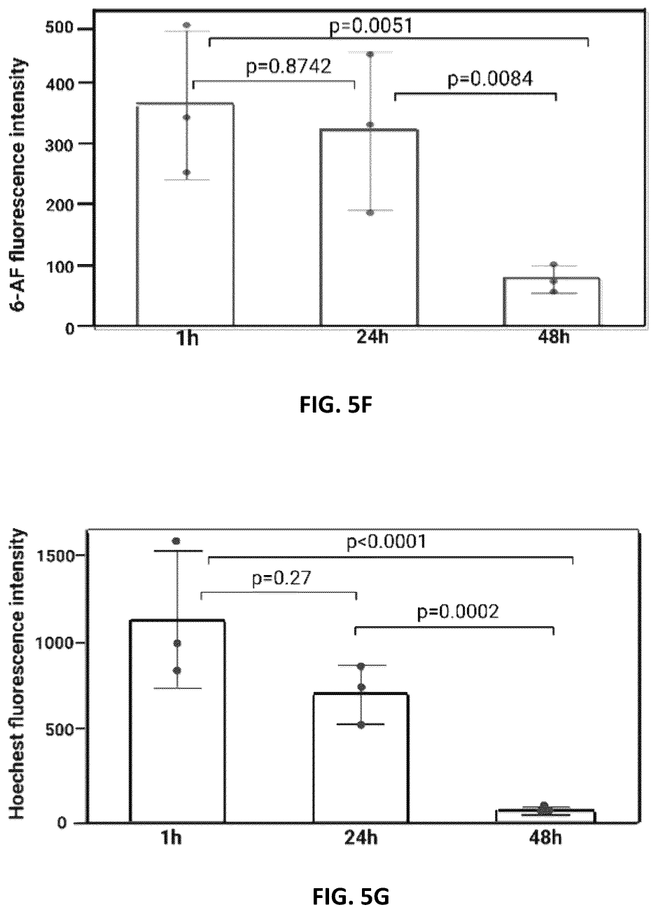

This procedure was developed based on the ability of Hoechst dye to bind to DNA through a non-covalent reaction. Hoechst dye can bind to the major and minor grooves of the DNA double helix. The major groove is the wider space between the two strands of the DNA, and the minor groove is the narrower space on the opposite side. The binding to the major and minor grooves occurs through hydrogen and van derWaals interaction. Hoechst exhibits strong binding and high affinity to the minor groove of the double-helical DNA sequences that are rich in AT base pairs, and can serve as a fluorescence dye for the characterization of DNA plasmid location in plant cells. The blue color emitted by Hoechst dyes comes specifically through Hoechst binding to the minor groove (FIG. 5a, scheme).

The CNPs/Hoechst/DNA plasmid complex is visually represented both schematically and through CLSM images in FIG. 5b. The 6-AF channel clearly shows the CNPs, and the blue channel exhibits the blue emission of the Hoechst dye bound to the DNA plasmid at 461 nm. The specific physical binding between the Hoechst dye and the DNA plasmid, is evident in the blue emission observed at 461 nm in the Hoechst dye's channel. In the merged image, the presence of Hoechst/DNA on the top of CNPs is clearly depicted, indicating the successful formation of CNPs/Hoechst/DNA plasmid conjugates. This configuration allows for effective tracking of DNA using CNPs as carriers within the leaves.

The CNPs/Hoechst//DNA plasmid complex was introduced into N. Benthamiana leaves using infiltration. FIG. 5c shows a schematic representation of the process that can occur in the N. benthamiana cells following the infiltration of the CNPs/Hoechst/DNA plasmid complex. Upon infiltration, the complex can be found both in the plant's cytoplasm and nucleus, indicating successful delivery. In the cytoplasm, both the CNPs and the DNA can undergo degradation by cellular enzymes but in the nucleus, the DNA may either be degraded by nuclear enzymes or turned into mRNA, leading to the production of the DsRed protein.

To assess the protective capability of CNPs in safeguarding DNA inside the cells, the CNPs/Hoechst/DNA plasmid complex was studied at three different times: 1, 24, and 48 h post infiltration. Confocal fluorescence microscopy images were obtained to capture the location of the complex within the cells. The testing was conducted on three different leaves from different plants. At 1 h post-infiltration, the image exhibited the CNPs in the green channel and the Hoechst in the blue channel, both colocalized in the same cellular regions, indicating that the complex successfully reached both the plant nucleus and the cytoplasm without disintegrating along the way. Even after 24 h, we could still see the Hoechst signal in the same part as the CNPs, conforming to the continued presence of the complex in the targeted areas. After 48 h, however, the Hoechst signal was localized in the nucleus, and at the same time, CNPs exhibited almost complete degradation. Note that the extent of degradation varied between the samples after 48 h, with some samples showing a range from partial to complete degradation.

CNPs were observed in the nucleus or were still visible in the cytoplasm (FIG. 5c-confocal). These results suggest that CNPs served as effective carriers, successfully delivering the DNA plasmid to the nucleus and to the cytoplasm within 24 h. Over time, however, the CNPs started to degrade, affecting their ability to maintain the bounded DNA protection against degradation.

Forty-eight hours post-infiltration, the presence of the Hoechst in the nucleus can be attributed to the possibility that Hoechst molecules were bound to the N. benthamiana DNA after their potential release from the degraded DsRed plasmid. The absence of DsRed protein expression suggests that the presence of Hoechst molecules may affect the plasmid's transcription or translation processes, as DNA dyes like Hoechst are recognized for their potential to induce DNA damage, particularly during DNA replication.

We characterized the cellular distribution of the CNPs/Hoechst/DNA plasmid complex in the cells by analysis of the Z-stack data collected with confocal microscopy. We merged the confocal data into 3D micrographs by Fiji-ImageJ software. The micrograph captured at 1 h post-infiltration confirms the presence of the CNPs/Hoechst/DNA plasmid complex within the cells (FIG. 5d).

FIG. 5e shows a quantitative analysis of colocalization dynamics of the green fluorescent signal attributed to the 6-AF/CNPs and the blue fluorescent signal attributed to the Hoechst/DNA at 1, 24, and 48 h post-infiltration. The analysis is based on Manders' coefficient (m1 and m2) calculated using the JACoP plugin in the Fiji-ImageJ software. The Manders' coefficient serves as a valuable tool for assessing the degree of colocalization between different fluorophores, which typically represent a given molecule. This coefficient provides a numeric value between 0 and 1, where 0 signifies no colocalization and 1 indicates complete colocalization. The Manders value is independent of different intensities between the evaluated channels, which assisted the current analysis because the Hoechst/DNA signal obtained was higher than the CNPs one. The ml value represents the fraction of the green channel (CNPs) that overlaps with the blue channel (Hoechst/DNA) and is calculated using Equation (1).

$$m 1 = Σ ( Gi ) coloc Σ ( Gi ) total \qquad ( 1 )$$

where (G i)coloc is defined as the intensity of the green fluorophore in pixels. Similarly, m2 represents the fraction of the blue channel (Hoechst/DNA) that overlaps with the green channel (6-AF/CNPs) and is calculated using Equation (2).

$$m 2 = Σ ( Bi ) coloc Σ ( Bi ) total \qquad ( 2 )$$

where (B i)coloc is defined as the intensity of the blue fluorophore in pixels.

At both 1 and 24 h, ml shows complete colocalization, meaning that 6-AF/CNP overlaps with the Hoechst/DNA in all shared colocalized pixels, which confirms the electrostatic adsorption of the DsRed plasmid onto the 6-AF/CNPs after their inter localization in the cells. The findings are supported by the insignificant difference in ml between 1 and 24 h (p-value of 1.0), suggesting that CNPs protect the DNA from degradation during this time. But after 48 h, as CNPs degrade, ml decreases compared to 1 h (pvalue of 0.1093) and 24 h (p-value of 0.1089). Furthermore, there was no significant decrease in m2 between 1 and 24 h (p-value of 0.2072), but there was a significant decrease in m2 between 1 and 48 h (p-value of 0.0059) and between 24 and 48 h (p-value of 0.00565). Furthermore, m2 was consistently lower than ml across all the time points. This can be attributed to the smaller surface area of the DNA than of the CNPs, leading to reduced colocalization with the CNPs and indicating a strong relationship between CNPs and DNA.

The green (CNPs) and blue (Hoechst/DNA) fluorescent signals observed in the cells at different time intervals were quantified by Fiji-ImageJ software based on the method developed by Duncan et al., as shown in FIG. 4f,g, respectively. The green fluorescence intensity started at a value of 396.2±123.1 1 h post-infiltration, decreased insignificantly to 326.7±131.4 at 24 h, then decreased to a minimal level of 81.0±23 at 48 h post infiltration (FIG. 5f). The blue fluorescence intensity shown in FIG. 5g started at a value of 1140.0±387.9, decreased insignificantly to 716.7±167.4 at 24 h, and decreased to a minimum level of 58.5±21.5, 48 h post-infiltration. These results also confirm the electrostatic adsorption of the DsRed plasmid onto the 6-AF/CNPs after their inter localization in the cells.

To quantitatively investigate the internalization of the CNPs/6-AF (pH 4.5, +13.0±4.9 mV, 214±82 nm) with the DsRed plasmid (avoiding any Hoechst-related impact on gene expression) into N. benthamiana cells, we prepared electrostatic conjugates of CNPs/6-AF/DsRed plasmid (24 h reaction). The CNPs/6-AF/DsRed plasmids were infiltrated into the leaves and the extract was analyzed by RT-PCR. This process involved three distinct CNPs: DsRed plasmid ratios: 1:003, 1:006, and 1:0.009.

We extracted total RNA from individual leaves infiltrated with three different CNPs/6-AF: DsRed plasmid ratios and from uninfiltrated control leaves (N. Benthamiana). The leaf RNA was subjected to DNase treatment to eliminate residual plasmid DNA that might lead to false positive amplification and reverse transcription reactions in the preparation of the cDNA templates.

FIG. 5h shows that all the infiltrated and control leaves express GAPDH, a constitutively expressed gene from the conserved glycolysis metabolic pathway. This validates the quality and integrity of the prepared cDNA samples by amplifying a 125 bp product from the leaves but not from the plasmid. FIG. 5i shows that expression of DsRed2 is detectable in N. benthamiana leaves 24 h post-infiltration. RT-PCR analysis using specifically designed primers inside the DsRed2 open reading frame clearly shows an amplified product with the expected size of 511 bp when the plasmid is used as a template (DsRed). Only the leaves that underwent infiltration contained DsRed2 mRNA, as evidenced by the amplification bands, in contrast to the control leaves that yielded no signal. These results is another major confirmation for successful expression of the DsRed plasmid which was delivered to the N. Benthamiana by the studied CNPs.

### Example 4

### Delivery of DsRed Plasmid into N. benthamiana Cells by CNPs

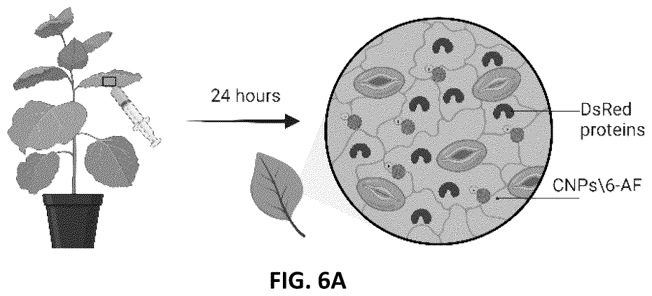

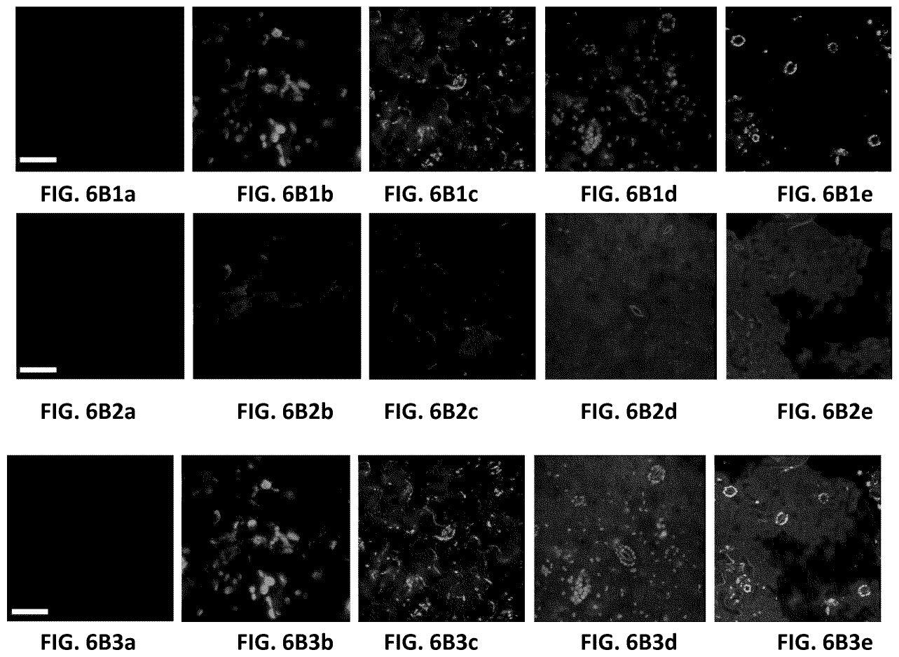

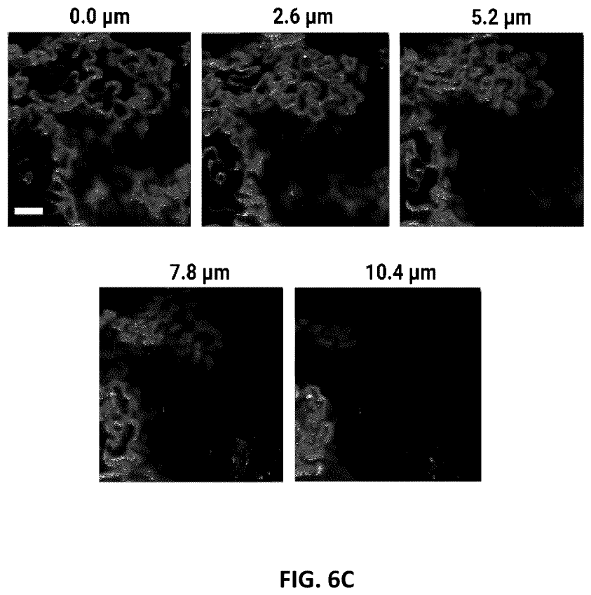

The CNPs/6-AF (pH 4.5, +13.0±4.9 mV, 214±82 nm), which were successfully internalized in the N. benthamiana cells at 2 mg mL-1, were investigated for their ability to deliver DNA to the plant cells. To this end, CNPs/6-AF/DsRed plasmid (pH 4.5, +13.0±4.9 mV, 214±82 nm) electrostatic conjugates were prepared in a 24 h reaction at four different CNPs: DsRed plasmid ratios of 1:0.001, 1:0.003, 1:0.005, and 1:0.01. To study the capability of the CNPs/6-AF (pH 4.5, +13.0±4.9 mV, 214±82 nm) to deliver DNA into the N. benthamiana cells and to characterize its expression rate, we detected the expressed DsRed protein by confocal microscopy, followed by the examination of control experiments associated with this system. The CNPs/6-AF/DsRed plasmid (pH 4.5, +13.0±4.9 mV, 214±82 nm) was infiltrated into N. benthamiana leaves at the different CNPs: DsRed plasmid ratios. The infiltration procedure was carried out by picking the abaxial side of the leaf (FIG. 6a) using a pipette tip. Quantitative measurement of the DsRed construct in infiltrated leaves by qRT-PCR shows that an increase in relative expression coincide with higher plasmid ratios. Leaves with CNP: DsRed plasmid ratio of 1:0.009 exhibit a DsRed expression level that is more than 4 times higher than their 1:0.003 counterparts (FIG. 6b). The infiltrated leaves were investigated by confocal fluorescence microscopy 24 h post infiltration. The z-stack micrographs of the systems are shown in FIG. 6c. A green fluorescent signal (emission of 500-540) corresponding to CNPs/6-AF, and a red fluorescent signal (emission of 565-640 nm) corresponding to the DsRed protein in the N. benthamiana cells were observed in the cytoplasm of all the CNPs: DsRed plasmid ratios (FIG. 6c). These results conclusively confirm the successful delivery of the DsRed plasmid by the CNPs/6-AF (pH 4.5, +13.0±4.9 mV, 214±82 nm) to N. benthamiana cells, followed by its expression. The negative control did not exhibit a red fluorescent signal, confirming that the fluorescence (green and red channels) in the experimental systems indeed corresponded to the DsRed and was not an autofluorescence of any component in the cell.

In addition, the results qualitatively showed a relatively low expression rate of the CNP: DsRed plasmid ratios of 1:0.001 and 1:0.003, with the expression rate at 1:0.005 and 1:0.01 exhibiting a much stronger and more homogeneous red fluorescent signal (FIG. 6c). Therefore, we studied further the CNPs: DsRed plasmid ratio of 1:0.01, which qualitatively presented the highest DsRed expression. FIG. 6c shows different views of the red channel Z-stack series attributed to the CNPs: DsRed plasmid ratio of 1:0.01. The results show that successful gene expression occurred throughout the cytoplasm.

### Example 5

### Internalization and Successful Gene Expression Versus Time of CNP/DsRed Plasmid Conjugates in the N. benthamiana Cells

Internalization versus time of the CNPs/6-AF/DsRed plasmid (pH 4.5, +13.0±4.9 mV, 214±82 nm) conjugates and the successful gene expression versus time of the DsRed plasmid were investigated by infiltration of the CNPs into N. benthamiana leaves and characterization of the leaves by fluorescent confocal microscopy at different time intervals post-infiltration (1 to 72 h).

The CNP concentrations in the dispersion and the CNP: DsRed plasmid ratios were at optimal values of 2 mg mL-1 (FIG. 3) and 1:0.01 (FIG. 6), respectively.

We monitored the localization of CNPs inside the cell by their green fluorescence. Even at 1 h, the CNPs could be seen inside two nuclei in the guard cells of the stomata. At 48 h, they were observed mainly in the nucleus, and at 72 h post-infiltration, the CNPs had fully degraded. We monitored the expression of the DsRed plasmid by the fluorescence of the DsRed protein. The first appearance of the red signal emitted by the DsRed protein was observed 24 h post-infiltration and remained for 48 h.

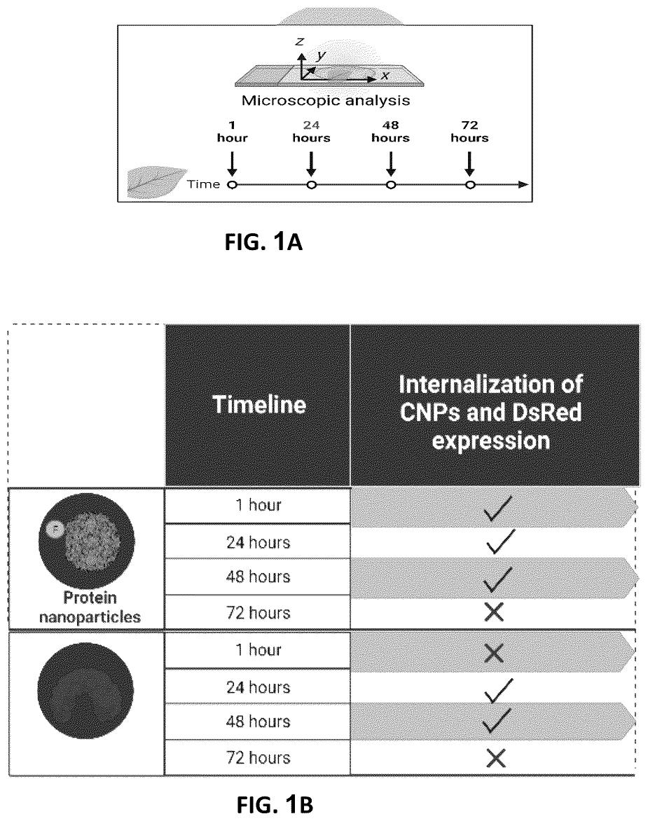

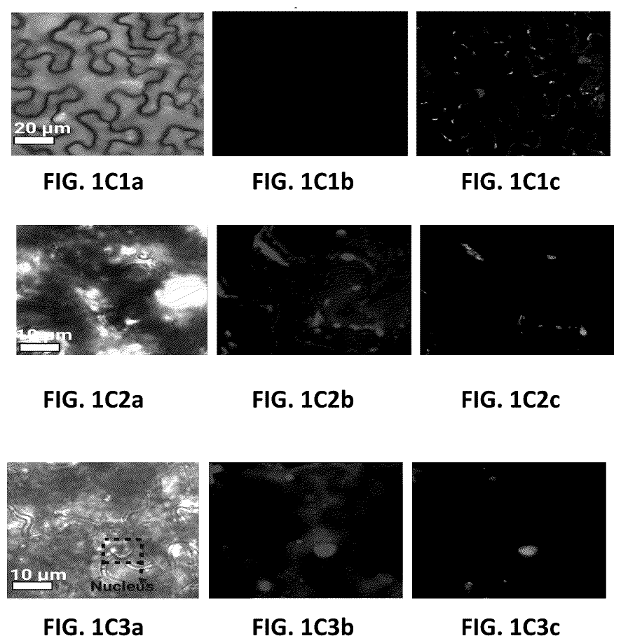

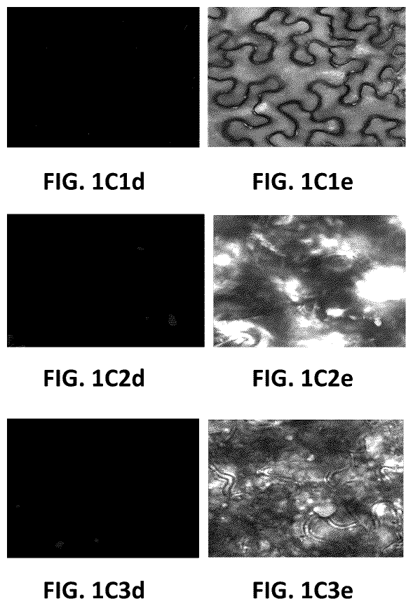

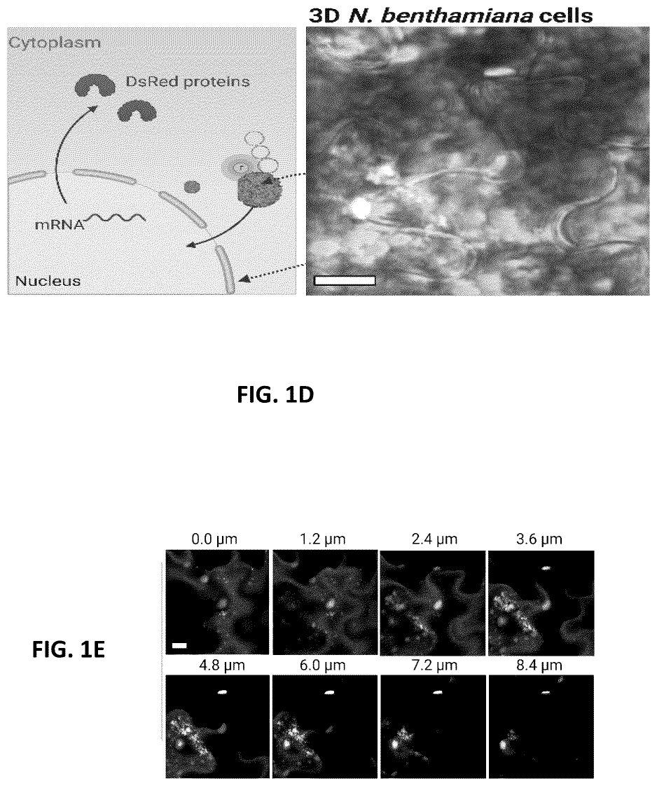

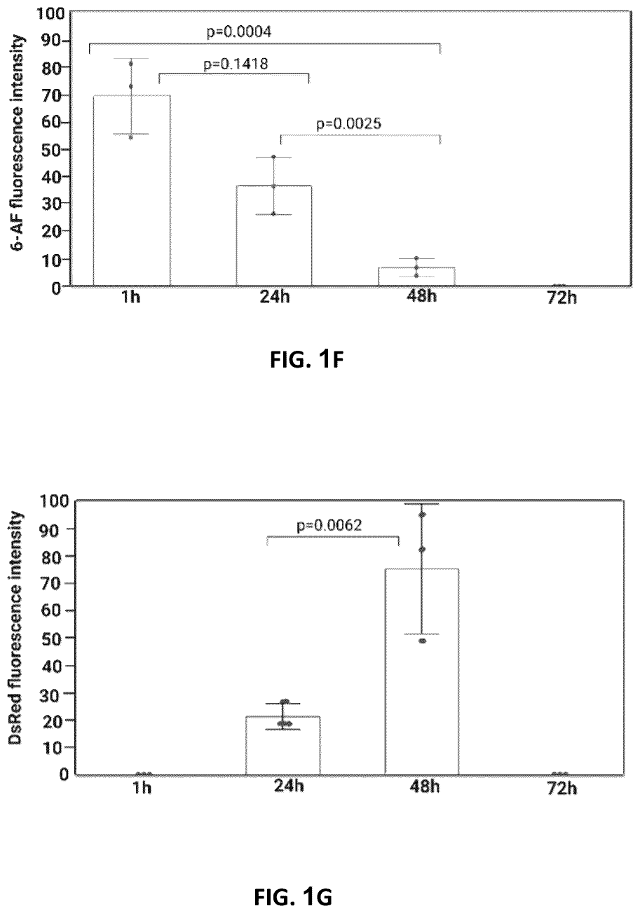

The red (DsRed protein) and the green (CNPs) fluorescent signals observed in the cells at different time intervals were quantified by Fiji-ImageJ software using the method developed by Duncan et al. are shown in FIG. 1f,g, respectively. The green fluorescence intensity started at a value of 65.6±14.7, 1 h post-infiltration, decreased significantly to 36.0±10.3 at 24 h, and reached 6.7±3.2, 48 h post-infiltration. Seventy-two hours post-infiltration, the green fluorescence intensity dropped to zero, demonstrating the biodegradability of the CNPs, a clear added value of a protein-based nanocarrier, compared to other nanocarriers (FIG. 1f). The expression level of DsRed is shown in FIG. 1g. The red fluorescence intensity was zero at 1 h, and reached a maximum level of 75.2±23.7 48 h post-infiltration, although the CNPs had been reduced by 90% at that time. This result indicates that most of the DsRed plasmid was desorbed from the CNPs in the early stages of the internalization and before the degradation of the CNPs by the several protection mechanisms of the plant. Naked DNA in the cell usually undergoes fast degradation by cellular nuclease activity. In the present study, however, the adsorption of the DNA (DsRed plasmid) onto the CNPs has protected it from the nucleases, as also shown by Demirer et al. After overcoming the nucleases, the nanocarrier must escape the endocytic pathway because only then is penetration into the nucleus possible. The schema in FIG. 1d shows the delivery of DsRed plasmid into a plant cell by the CNPs followed by its expression.

FIG. 1d shows 3D visualization by confocal micrographs, 48 h after infiltration, of all four channels (red, green, another red that converted to blue, and a bright field). To investigate the expression of the DsRed throughout the cells, we analyzed different planes (single-layer micrographs) of the z-stack, which are shown in FIG. 1e. The data indicate that after 48 h, both the CNPs and the expressed DsRed protein were located inside the nucleus, as confirmed by the yellow resulting from the colocalization of the red fluorescent emission of the DsRed plasmid with the green fluorescent emission of the CNPs.

To this end CNPs/6-AF/DsRed plasmid (pH 4.5, +13.0±4.9 mV, 214±82 nm) electrostatic conjugates were infiltrated into N. benthamiana plants for gene expression and characterized by confocal fluorescence microscopy. The confocal microscopy results indicated successful DNA delivery to the cells of N. benthamiana plants. A red signal was detected inside the cells indicating that a DsRed expression had taken place. The successful delivery was obtained by CNPs/6-AF/DsRed plasmid (pH 4.5, +13.0±4.9 mV, 214±82 nm) electrostatically attached to the DsRed plasmid. Our nanocarriers demonstrated successful DNA delivery and gene expression without surface modification. The zeta potential of the CNPs was tuned by alteration of the pH. The current work presents DNA delivery by protein nanoparticles without any further modification, opening up the possibility of developing a highly efficient, practical, and cost-effective DNA delivery system for intact plant cells that complies with regulatory demands. The optimal conditions for DNA delivery were 2 mg mL-1 CNPs/6-AF/DsRed plasmid (pH 4.5, +13.0±4.9 mV, 214±82 nm) at a CNP: DsRed plasmid ratio of 1:0.01.

Confocal microscopy results showed that 1 h post-infiltration, the CNPs could be seen in the cytoplasm and in the nucleus, and that at 72 h post-infiltration, the CNPs had fully degraded. The expression of the DsRed plasmid was monitored by the fluorescence of the DsRed protein. The first appearance of the emitted red signal of the DsRed protein was observed 24 h post-infiltration and remained for 48 h. In addition to the confirmation by confocal microscopy, the successful gene expression was also confirmed by RT-PCR and qRT-PCR.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising a plurality of particles, wherein:
each of the plurality of particles comprises a casein nanoparticle and a polynucleotide bound to said casein nanoparticle;
said plurality of particles are characterized by:
(i) an average zeta potential below 0 mV when measured at a pH of about 4.5; and by
(ii) an average particle size ranging between 20 nm and about 300 nm.

2. The composition of claim 1, wherein said plurality of particles is characterized by an average zeta potential of between −1 and −50 mV.

3. The composition of claim 2, wherein said plurality of particles s characterized by an average particle size ranging between about 200 nm and about 250 nm, as determined by DLS; and wherein said casein nanoparticle is a non-crosslinked particle.

4. The composition claim 1, wherein a weight per weight ratio between said polynucleotide and said casein nanoparticle within the composition is between 0.1:1 and 2:1, and wherein said plurality of particles consists essentially of casein and the polynucleotide, including any salt thereof.

5. The composition of claim 1, wherein said bound is via electrostatic interactions.

6. The composition of claim 1, wherein said polynucleotide comprises RNA, DNA, or both; and wherein said polynucleotide is bound to an outer surface of the casein nanoparticle.

7. The composition of claim 6, wherein said DNA comprises an expression vector or a plasmid.

8. The composition of claim 1, wherein said plurality of particles is characterized by an average zeta potential of between −5 and −30 mV.

9. The composition of claim 1, being an agricultural composition further comprising an agriculturally acceptable carrier.

10. The composition of claim 1, being formulated for administration by spraying, drenching, dipping, soaking, injecting, or any combination thereof.

11. The composition of any one of claim 1, wherein a concentration of said plurality of particles within the composition is between 0.1 and 10 mg/ml.

12. A method for delivering a polynucleotide into a cell of a plant, comprising contacting said plant or a part of said plant with an effective amount of the composition of claim 1, thereby delivering the polynucleotide to the cell of said plant.

13. The method of claim 12, wherein said contacting is by a method selected from: injecting, spraying, drenching, dipping, soaking, or any combination thereof.

14. The method of claim 13, wherein said method is for modifying the expression of at least one gene within the plant.
